(19) United States
(12) Patent Application Publication
(10) Pub. No.: US 2003/0165853 A1
(43) Pub. Date: Sep. 4, 2003

(54) Antisense imaging of gene expression of the brain in vivo

(75) Inventors: William M. Partridge, Pacific Palisades, CA (US); Ruben J. Boado, Agoura Hills, CA (US)

Correspondence Address: QUINE INTELLECTUAL PROPERTY LAW GROUP, P.C., P O BOX 458, ALAMEDA, CA, 94501, US

(73) Assignee: The Regents of the University of California Office of Technology Transfer

(21) Appl. No.: 10/005,996

(22) Filed: Dec. 3, 2001

Related U.S. Application Data
(60) Provisional application No. 60/250,990, filed on Dec. 4, 2000.

(51) Int. Cl. A61K 51/00; C12Q 1/68

(52) U.S. Cl. 435/6; 424/1.49; 424/1.73

(57) ABSTRACT

This invention provides imaging reagents for the detection of a gene or gene expression product (e.g. mRNA) in a brain cell in vivo. Preferred reagents comprise a detectable label attached to a first nucleic acid that specifically hybridizes to the gene or to a nucleic acid transcribed from the gene. The first nucleic acid is linked to a targeting ligand that is capable of binding a receptor on a cell comprising the blood brain barrier and crossing said blood brain barrier.

### CROSS-REFERENCE TO RELATED APPLICATIONS

This application claims benefit of and priority to U.S. Ser. No. 60/250,990, filed on Dec. 4, 2000, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention relates to the field of imaging. In particular, this invention relates to a class of imaging reagents that are capable of crossing the blood brain barrier and providing in vivo imaging of gene expression in the brain.

### BACKGROUND OF THE INVENTION

With the completion of sequencing of the human genome and the ongoing sequencing of other genomes, ever increasing amounts of genetic information are becoming available in various databases. This will lead rapidly to the identification of genetic mutations that give rise to cancer and other chronic diseases of the brain and other organs. The presence of a gene mutation in a given individual can be determined with a simple blood test. However, the blood test merely tells the individual that they have the gene, not that the gene is activate at that point in time in their life. It could be many years before the gene becomes active and causes the cancer or chronic disease. Therefore genetic counseling is greatly limited in guiding medical therapy because present methodology does not indicate when a given gene is being expressed. It would be highly advantageous to have an imaging modality or brain scan that allowed for detection of the active expression of a given gene within the brain.

The ability to image active gene expression in the brain in vivo would also be advantageous in determining subsets of a given population of patients that are most appropriate for a given type of drug therapy. For example, within a given highly heterogenous disease such as schizophrenia, a certain subset of the population may overexpress a certain drug receptor. In this case, these patients may be best suited for a certain line of therapy, whereas other subsets of the population may not express the gene and may be better suited for other lines of drug therapy.

The imaging of gene expression has been performed for many years in autopsy brain sections in vitro, and this is based on the complementary binding of nucleic acid polymers. That is, an antisense oligodeoxynucleotide (ODN) binds to the sense mRNA in a brain tissue/cell to form a nucleic acid duplex, and this hybridization is highly sequence specific. Prior to this invention, however, it is believed that such imaging of gene expression in brain tissue has been limited to in vitro methods and thus has been impractical as a prognostic/diagnostic methodology.

### SUMMARY OF THE INVENTION

This invention provides novel imaging reagents that are capable of crossing the blood-brain barrier in vivo, and entering brain cells in sufficient concentration that they can be readily detected using standard detection methods. In preferred embodiments, the imaging reagent comprises a nucleic acid that specifically hybridizes to a gene or gene product (e.g. an mRNA) attached to a targeting ligand that is capable of binding a receptor on a cell comprising the blood brain barrier and crossing the blood brain barrier. The imaging reagent also bears a detectable label, e.g. attached directly or indirectly to the nucleic acid (e.g. attached directly to the nucleic acid, attached to the targeting ligand, etc.).

Thus, in one embodiment, this invention provides an imaging reagent that labels a gene or gene product. The imaging reagent comprises a detectable label attached to a first nucleic acid that specifically hybridizes to the gene or to a second nucleic acid transcribed from the gene, where said first nucleic acid is linked to a targeting ligand that is capable of binding a receptor on a cell comprising the blood brain barrier and crossing the blood brain barrier. In preferred embodiments the targeting ligand is an antibody that specifically binds to a receptor on a cell comprising the blood brain barrier, or a substrate specifically bound by a receptor on cell comprising the blood brain barrier. Particularly preferred targeting ligands include, but are not limited to insulin, transferrin, insulin-like growth factor I (IGF-I), insulin-like growth factor II (IGF-II), basic albumin, leptin, and prolactin and analogues, derivatives or mimetics thereof or antibodies that bind to a receptor selected from the group consisting of an insulin receptor, a transferrin receptor, an insulin-like growth factor I (IGF-IR) receptor, and insulin-like growth factor II receptor (IGF-IIR), and a leptin receptor.

In certain embodiments, the first nucleic acid is linked to the targeting ligand by a linker or by an affinity tag (e.g. an affinity tag comprising a biotin and a molecule that binds to biotin). Particularly preferred affinity tags include a biotin and an avidin, a streptavidin, or an anti-biotin antibody. In certain embodiments the nucleic acid is modified to improve stability or facilitate entry into a cell. Particularly preferred nucleic acids include, but are not limited to peptide nucleic acids. The nucleic acid can be protected by the use of blocking/protecting groups. Thus, for example in certain embodiments, the carboxyl terminal of the peptide nucleic acid is amidated.

Detectable labels suitable for use in the reagents of this invention include, but are not limited to radioactive labels, magnetic labels, spin labels, enzymatic labels, colorimetric labels, and fluorescent labels. In certain embodiments, the nucleic acid is labeled with a radiolabeled amino acid (e.g. a tyrosine labeled with ¹²⁵I, a lysine labeled with ¹¹¹In, etc.).

Virtually any gene or gene product can be specifically targeted (labeled) using the imaging reagents of this invention. Preferred targets include a gene or mRNA that encodes a molecule selected from the group consisting of a receptor, and enzyme, a structural protein, and a transcription factor.

In some particularly preferred embodiments, the first nucleic acid is a peptide nucleic acid, the targeting ligand is an antibody (e.g. a monoclonal antibody, a single-chain antibody, etc.) that specifically binds to a receptor (e.g. a transferrin receptor, an insulin receptor, etc.) on a cell comprising the blood-brain barrier; and first nucleic acid is attached to the targeting ligand through an affinity tag. The imaging reagent comprises a radioactive label (e.g., a radiolabeled amino acid), or a magnetic label. In some preferred embodiments, the affinity tag is an affinity tag comprising a biotin. As indicated above, in certain embodiments, the carboxyl terminal of the peptide nucleic acid is amidated.

This invention also provides methods of use of the imaging reagents (e.g., methods of imaging expression of a gene or cDNA in a brain cell). The methods involve contacting the brain cell with an imaging reagent, (as described herein) and detecting the presence or quantity of a signal produced by the detectable label (on the reagent) in the brain cell where the presence or quantity of the label indicates the presence or quantity of a nucleic acid transcribed from said gene or cDNA.

The brain cell can be a cell in culture, however, in preferred embodiments, the cell is preferably a cell in vivo in a living organism. Preferred organisms include vertebrates, more preferably mammals (e.g. murine, rodent, largomorph, bovine, feline, canine, equine, non-human primate, human, etc.).

This invention also provides kits for imaging a gene or a gene product in a brain cell. Preferred kits include a container containing an imaging reagent comprising a detectable label attached to a first nucleic acid that specifically hybridizes to a second nucleic acid transcribed from the gene, where the first nucleic acid is linked to a targeting ligand that is capable of binding a receptor on a cell comprising the blood brain barrier and crossing said blood brain barrier. Other kits include a container containing a nucleic acid that specifically hybridizes to said gene or to a nucleic acid transcribed from said gene attached to an affinity tag; and a container containing a targeting ligand that is capable of binding a receptor on a cell comprising the blood brain barrier and crossing the blood brain barrier, where the targeting ligand is attached to an affinity tag such that when said nucleic acid is contacted to said targeting ligand the nucleic acid attaches to the targeting ligand by binding of the affinity tags (e.g. biotin/avidin link). In preferred kits the nucleic acid is a peptide nucleic acid labeled with a detectable label.

Definitions.

A “chimeric molecule” is a molecule comprising two or more molecules typically found separately in their native state that are joined together typically through one or more covalent bonds. The molecules may be directly joined or joined through a linker. Where the molecules are both polypeptides they may be joined through a peptide bond or a peptide linker forming a fusion protein.

A “fusion protein” refers to a polypeptide formed by the joining of two or more polypeptides through a peptide bond formed between the amino terminus of one polypeptide and the carboxyl terminus of another polypeptide. The fusion protein may be formed by the chemical coupling of the constituent polypeptides or it may be expressed as a single polypeptide from nucleic acid sequence encoding the single contiguous fusion protein. A single chain fusion protein is a fusion protein having a single contiguous polypeptide backbone.

A “spacer” or “linker” as used in reference to a fusion protein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

A “spacer” or “linker” as used in reference to a chemically conjugated chimeric molecule refers to any molecule that links/joins the constituent molecules of the chemically conjugated chimeric molecule.

The terms “binding partner”, or a member of a “binding pair”, or “cognate ligand” refers to molecules that specifically bind other molecules to form a binding complex such as antibody/antigen, lectin/carbohydrate, nucleic acid/nucleic acid, receptor/receptor ligand (e.g. IL-4 receptor and IL-4), avidin/biotin, etc.

The term ligand is used to refer to a molecule that specifically binds to another molecule. Commonly a ligand is a soluble molecule, e.g. a hormone or cytokine, that binds to a receptor. The decision as to which member of a binding pair is the ligand and which the “receptor” is often a little arbitrary when the broader sense of receptor is used (e.g., where there is no implication of transduction of signal). In these cases, typically the smaller of the two members of the binding pair is called the ligand. Thus, in a lectin-sugar interaction, the sugar would be the ligand (even if it is attached to a much larger molecule, recognition is of the saccharide).

The term “substrate for a receptor” refers to a non-antibody ligand that is bound by a receptor. In particularly preferred embodiments, the substrate for a receptor is the cognate ligand for that receptor, although the term also contemplates the use of mimetics or derivatives of such cognate ligands.

The terms “epitope tag” or “affinity tag” are used interchangeably herein, and used refers to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, His₆ bound by Ni—NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO: 1) epitope, c-myc antibodies (available from Sigma, St. Louis), the HNK-1 carbohydrate epitope, the HA epitope, the HSV epitope, the His₄, His₅, and His₆ epitopes that are recognized by the His epitope specific antibodies (see, e.g., Qiagen), and the like. In addition, vectors for epitope tagging proteins are commercially available. Thus, for example, the pCMV-Tag1 vector is an epitope tagging vector designed for gene expression in mammalian cells. A target gene inserted into the pCMV-Tag1 vector can be tagged with the FLAG® epitope (N-terminal, C-terminal or internal tagging), the c-myc epitope (C-terminal) or both the FLAG (N-terminal) and c-myc (C-terminal) epitopes.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” refer to at least two nucleotides covalently linked together. A nucleic acid of the present invention is preferably single-stranded or double stranded and can contain phosphodiester bonds, although in certain preferred embodiments, as outlined below, nucleic acids are included that may have alternate backbones, comprising, for example, phosphoramide (Beaucage et al. (1993) Tetrahedron 49(10):1925); Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}—V_{L} heterodimer which may be expressed from a nucleic acid including V_{H}— and V_{L}— encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No: 5733743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8:1323-1331).

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions the specified ligand, antibody, or nucleic acid binds to its particular “target” molecule and does not bind in a significant amount to other molecules present in the sample. In particularly preferred embodiments, a first nucleic acid specifically binds a second nucleic acid if the first nucleic acid is complementary to all or to a part of said second nucleic acid.

The terms “hybridizing specifically to” and “specific hybridization” and “selectively hybridize to,” as used herein refer to the binding, duplexing, or hybridizing of a nucleic acid molecule preferentially to a particular nucleotide sequence. The specific binding can be under physiological “normal conditions” such as occur in a cell. The ability of a particular nucleic acid to specifically bind to another nucleic acid can be evaluated by evaluating hybridization under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, NY (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4x to 6×SSC at 40° C. for 15 minutes.

As used herein, a nucleic acid “derived from a nucleic acid” (e.g., an mRNA) refers to a nucleic acid for whose synthesis the mRNA or a subsequence thereof has ultimately served as a template. Thus, a cDNA reverse transcribed or RT-PCR'd from an mRNA, an RNA transcribed from that cDNA, a DNA amplified from the cDNA, an RNA transcribed from the amplified DNA, etc., are all derived from the mRNA and detection of such derived products is indicative of the presence and/or abundance of the original in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

### BRIEF DESCRIPTION OF THE DRAWINGS

FIGS. 1A and 1B illustrate a construct of this invention. FIG. 1A: The sequence of the antiluciferase antisense PNA is shown along with the biotin (bio) moiety at the amino terminus and the tyrosine (Y) and lysine (K) moiety at the carboxy terminus. There are five linkers (O) at both the near carboxy and near amino termini. The sequence of luciferase mRNA around the Methionine initiation codon (ATG) is shown. The luciferase (Luc) ORF(orf) was subcloned into a eukaryotic expression plasmid, designated clone 790, which was described previously (Boado and Pardridge (1998) Mol. Brain Res. 59: 109-113). The hygromycin selection gene, the SV40 promoter, the SV40 39 UTR, and the Epstein-Barr nuclear antigen (EBNA)-1 gene are shown. The SV40 39 UTR contains 200 nucleotides from the bovine (b) GLUT1 glucose transporter mRNA, which optimizes gene expression through mRNA stabilization (Id.). FIG. 1B: Antisense imaging agent is comprised of the OX26 mAb to the rat TfR, which is linked to SA, which binds the monobiotinylated PNA antisense agent. The PNA contains a Tyr and Lys residue at the amidated carboxy terminus to enable radiolabeling with 125-iodine on the Tyr residue or with 111-indium on the Lys residue.

FIG. 2 (Left) shows plasma radioactivity, expressed as percent of ID/ml, plotted vs. time after i.v. injection for the unconjugated PNA and the PNA conjugate, designated PNA/OX26-SA. Data are mean±SE (n=three rats). The right plot shows the percent of plasma radioactivity that is TCA precipitable.

FIG. 3 shows the percent of ID delivered per gram of tissue is for brain, heart, liver, and kidney. Data are mean 6 SE (n 5 three rats per group). The radiolabeled PNA was injected in one of two forms: (i) unconjugated, which is designated PNA in the figure, and (ii) as a conjugate of the PNA and the OX26/SA targeting system, which is designated PNA conjugate. Mean±SE (n=three rats).

FIGS. 4A: The uptake in either C6 cells or C6-790 cells is plotted against time for four different [¹²⁵I] tracers: the unconjugated antiluciferase PNA (designated PNA Luc), the unconjugated anti-rev PNA(designated PNA rev), the PNA Luc conjugated to OX26/SA, and the PNA rev conjugated to OX26/SA. Data are mean±SE (n=three rats) for each time point. FIG. 4B: Film autoradiography after SDS/PAGE of C6-790 cell extracts obtained at 4 (lane 1), 6 (lane 2), or 24 (lane 3) h incubation with either the PNA rev conjugate (Left) or the PNA Luc conjugate (Right). FIG. 4C: Pulse-chase experiment showing rate of loss of radioactivity from the cellular TCA precipitable fraction during a second 24-h period after the C6 cells or C6-790 cells were pulsed during an initial 24-h incubation with the labeled PNA Luc conjugate. Data are mean±SE (n=three dishes) for each time point. The radioactivity was significantly higher in the C6-790 cells at all time points (* indicates P<0.01, Student's t test).

FIG. 5 shows brain scans (left) and autopsy stains (right) are for three groups of rats designated A, B, and C. Group A rats received an i.v. injection of the [¹²⁵I] antiluciferase PNA bound to the conjugate of the OX26 mAb and SA, which is designated SA-mAb. Group B rats received [¹²⁵I]antiluciferase PNA without conjugation to SA-OX26. Group C rats received an i.v. injection of [¹²⁵I]anti-rev PNA bound to the SA-OX26 conjugate, which is designated SA-mAb.

FIGS. 6A: “Import-export” model showing the bi-directional movement of a conjugate of a peptide nucleic acid (PNA) antisense radiopharmaceutical and the rat 8D3 monoclonal antibody (MAb) to the mouse transferrin receptor (TfR). The bi-directional movement of the PNA-8D3 conjugate across either the blood brain barrier (BBB) or the brain cell membrane (BCM) is possible owing to the ability of the 8D3 MAb to access the endogenous transport pathways for transferrin (Tf), which exist at both cellular barriers. Access to the TfR pathways allows the PNA radiopharmaceutical to move between the blood and the intracellular compartment of the target cell. FIG. 6B: Conjugation of the PNA to the 8D3 MAb to the TfR creates a bifunctional molecule that both accesses the TfR for transport between tissue compartments and binds to a target mRNA based on the sequence specificity of the nucleotide residues of the PNA radiopharmaceutical. The PNA has a biotin moiety at the amino terminus to allow for capture by a conjugate of the 8D3 MAb and streptavidin (SA) and has carboxyl terminal tyrosine (Tyr) or lysine (Lys) residues to allow for radiolabeling with [¹²⁵I] or [¹¹¹In], respectively.

FIG. 7A: The nucleotide sequence (in a 5′ to 3′ orientation) (SEQ ID NO: 2) of the HD-PNA is bordered on the amino terminus by a biotin (bio) residue and by tyrosine (Y) and lysine (K) residues at the carboxyl terminus. There are five linkers (designated O) flanking the nucleotide sequence. The complementary nucleotide sequence of the HD target mRNA (in a 5′ to 3′ orientation) is shown and the methionine initiation codon (ATG) is underlined (SEQ ID NO: 3). The HD exon 1 sequence is downstream of the T3 RNA polymerase promoter which allows for in vitro transcription of HD exon 1 mRNA. FIG. 7B: Combined in vitro transcription/translation assays resulted in the formation of ³H-labeled exon 1 huntingtin protein that was precipitated by trichloroacetic acid (TCA). The translation of the HD exon 1 protein was inhibited in a dose response by either a PO—ODN (III) or by the PNA. FIG. 7C: The RNase protection assay (RPA) demonstrates formation of an HD mRNA protected fragment following complete nuclease digestion, owing to hybridization of the biotinylated HD PNA to the huntingtin exon 1 mRNA (lane 2). The conjugation of the antisense PNA to the MAb-SA transport vector does not inhibit the hybridization of the PNA to the target mRNA, based on the formation of the RNase protected oligonucleotide shown in lane 4. Conversely, no protected fragment is observed following mixing of an anti-luciferase (Luc) PNA with the HD RNA, either in an unconjugated form (lane 3) or conjugated to the MAb-SA vector (lane 5). BPB=bromophenol blue.

FIG. 8 illustrates the BBB permeability-surface area (PS) product, the area under the plasma concentration curve (AUC), and the brain uptake, expressed as % of injected dose (ID) per gram (g) brain, and these parameters are shown for either unconjugated [¹²⁵I]-PNA or [¹²⁵I]-PNA/8D3 conjugate. Data are mean±S.E. (n=3 mice per group). The units of the PS product are μL/min/g and the units of the 60-minute plasma AUC are %ID^{·} min/mL.

FIG. 9 shows the brain uptake, expressed as % of injected dose (ID) per gram (g) brain, at 1, 2, 4, and 6 hours after intravenous injection of the [¹²⁵I]-PNA/8D3 conjugate. Data are mean±S.E. (n=3 mice per group). Linear regression analysis yielded the intercept (Int.) and slope values that are shown. The PNA/8D3 conjugate underwent export from brain back to blood with a t_{1/2} of 4.3±0.5 hours.

FIG. 10A: Quantitative autoradiography (QAR) of 20 μm frozen sections of brain taken from 3 different littermate control mice or 3 different transgenic mice that were sacrificed 6 hours after a single intravenous injection of the [¹²⁵I] PNA/8D3 conjugate. FIG. 10B: QAR of the 20 μm thick [¹²⁵I]-microscale standard strips is shown in the inset. The integrated density for each standard is plotted versus the known radioactivity for the standard. FIG. 10C: The integrated density obtained from scanning the autoradiograms of the brain sections taken from either the littermate control mice or the transgenic mice (Panel A) was converted into measurements of organ radioactivity (μCi/g) based on the standard curve (FIG. 10B). The data indicate there is more than a 3-fold increase in sequestration of brain radioactivity at 6 hours after intravenous injection in the transgenic mice as compared to the littermate control mice. Data are mean±S.E., n=3 mice per group.

### DETAILED DESCRIPTION

This invention provides methods and compositions useful for imaging gene presence and/or expression level, and/or the amount of an antisense pharmaceutical in the brain, particularly of a brain in vivo. In preferred embodiments, the compositions comprise an imaging reagent comprising a nucleic acid that specifically hybridizes to a gene or gene product (e.g. an mRNA) attached to a targeting ligand that is capable of binding a receptor on a cell comprising the blood brain barrier and crossing the blood brain barrier. The imaging reagent also bears a detectable label, e.g. attached directly or indirectly to the nucleic acid (e.g. attached directly to the nucleic acid, attached to the targeting ligand, etc.).

The targeting ligand facilitates transport of the imaging reagent across the blood brain barrier. It was a surprising discovery of this invention that the imaging reagent is transported both across the blood brain barrier and across the cell membrane into brain cells with sufficient efficacy that it is possible to detect and/or quantify the imaging reagent in brain cells even where the administration is a systemic administration.

Thus, in certain embodiments, the methods of this invention involve administering the imaging reagent to a living organism (e.g. a mammal) and then detecting the presence or quantity of a signal produced by the detectable label in brain cell(s) of the organism, where the presence or quantity of the label indicates the presence or quantity of a nucleic acid transcribed from a particular targeted gene or cDNA. Because the imaging reagent is transported across the blood brain barrier there is no requirement for direct infusion into brain tissue (e.g. through cannulae) and the reagent can be administered through more conventional and convenient routes (e.g., oral administration, intravenous or subcutaneous injection, etc.).

Moreover, having demonstrated herein, that it is possible to deliver antisense molecules for imaging purposes across both the blood brain barrier and the cell membrane in sufficient quantity to monitor levels of gene expression, one of skill will appreciate that the same methods can be utilized to specifically modulate or inactivate expression of one or more target genes (e.g. using an antisense molecule, ribozyme, RNAi, etc. instead of the labeled nucleic acid).

In preferred embodiments, the methods of this invention utilize a construct (e.g., an imaging reagent) comprising a detectable label attached to a nucleic acid that can specifically bind to (hybridize to) a target nucleic acid found in a brain cell. The nucleic acid is also attached, directly or indirectly through a linker, to a targeting ligand that binds to a receptor on a cell comprising the blood brain barrier (BBB). Preferred targeting ligands bind to the receptor and are transported across the blood brain barrier. It was a surprising discovery of this invention that when such targeting ligands are coupled to a nucleic acid, the nucleic acid is not only transported across the blood brain barrier, but is also transported intact into brain cells where the nucleic acid is capable of hybridizing to a target nucleic acid (e.g. an mRNA); and inactivating or labeling that target nucleic acid. In preferred embodiments, the nucleic acid comprising the construct is an antisense nucleic acid and in particularly preferred embodiments, the nucleic acid is a peptide nucleic acid.

I. Targeting Ligand.

A wide variety of targeting ligands can be used in the reagents of this invention. Preferred targeting ligands are those that specifically bind a receptor on a cell comprising the blood-brain barrier, and as used herein, the term targeting ligand refers to a molecule that specifically binds or is bound by such a receptor. Particularly preferred targeting ligands are transported, by means of the receptor, across the blood-brain barrier (e.g. by transcytosis).

Preferred targeting ligands include, but are not limited to ligands that are specifically bound by the receptor (e.g. receptor substrates (cognate ligands)) and receptor-specific antibodies.

A) Receptor Specific Liands and Mimetics.

In certain embodiments, the targeting ligands include ligands that are capable of being bound by receptors and transported across the blood brain barrier. Such “transportable” polypeptides (or other ligands) are known to those of skill in the art (see, e.g. U.S. Pat. Nos. 4,801,575, and 6,287,792). Suitable transportable peptides include: insulin, transferrin, insulin-like growth factor I (IGF-I), insulin-like growth factor II (IGF-II), basic (cationized) albumin, avidin streptavidin or an avidin derivative/analogue, and prolactin.

Transferrin is an 80K glycoprotein that is the principal iron transport protein in the circulation. Transferrin is also a protein that is enriched in the cerebrospinal fluid (CSF). Transferrin is widely available and may be purchased or isolated from blood or CSF by well-known procedures.

Insulin, IGF-I and IGF-II are also commonly available. Insulin is available on a wide scale commercially and may also be recovered from natural sources by well-known techniques. IGF-I and IGF-II are available from commercial outlets such as Amgen or Peninsula Labs or they may be isolated from natural sources according to the procedure of Rosenfeld et al. (1982) J. Clin Endocrinol. Metab. 55, 434.

Basic albumin or cationized albumin typically has an isoelectric point higher than that of natural albumin (e.g. typical (pI) of about 8.5 as compared to a pI of about 3.9 for natural albumin). Cationized albumin, unlike natural albumin, enters the brain rapidly across the blood-brain barrier. Cationized albumin is prepared preferably by covalent coupling of hexamethylene-diamine (HMD) to bovine serum albumin according to Bergmann, et al. (1985) Endocrinology, 116:1729-1733. An exemplary synthesis is as follows: 10 ml of a 10% solution of albumin in water is slowly added to 60 ml of 2.0M HMD and the pH of the solution is adjusted to 6-7 with 1N HCl. After 30 minutes, 1 g of N-ethyl-N′-3-(dimethylaminopropyl)carbodiimide hydrochloride (EDAC) is added to activate the carboxyl groups of the albumin, followed by the addition of another 1 g EDAC 1 hour later. The pH is constantly adjusted to 6-7 with 0.2 N HCl. The solution is allowed to stand overnight with constant stirring. The next day the solution is dialyzed extensively against distilled water. This solution is then purified by chromatofocusing using the Pharmacia polybuffer exchanger 94 resin and the polybuffer 96 elution buffer.

Prolactin is a hormone which is secreted by the anterior pituitary. Prolactin is widely available commercially or it can be isolated from pituitary glands by well-known procedures.

Avidin is a cationic protein typically with an isoelectric point (pI) of about 10, (Green (1975) Adv. Protein Chem., 29: 85-133) owing to a preponderance of basic amino acids (lysine, arginine) relative to acidic amino acids (aspartic acid, glutamic acid) (Id). In contrast, the bacterial homologue of avidin, called streptavidin, which is 38% homologous with avidin, is a slightly acidic protein with a pI of about 5 to 6 (Green (1990) Meth. Enzymol., 184: 51-67). Like avidin, streptavidin binds biotin with extremely high affinity (Id). Streptavidin, a bacterial protein, is not glycosylated, and is capable of functioning as the avidin moiety, binding biotin. It has been shown that that avidin is taken up by tissues, such as brain, liver, and kidney by an absorptive-mediated endocytosis mechanism observed for other cationic proteins, such as histone or cationized albumin (. Kumagai et al. (1987) J. Biological Chem., 262: 15214-15219; Pardridge et al. (1989) J. Pharmacol. Exp. Ther., 251: 821-826).

Avidin is a 64,000 dalton homotetramer glycoprotein (Green (1975) Adv. Protein Chem., 29: 85-133), and has been administered to humans in large concentrations without untoward effects (Kaplan (1944) Am. J. Medical Science, 207: 733-743). Each 16,000 monomer of avidin contains a high-affinity binding site for the water-soluble vitamin biotin and the avidin tetramer binds four biotin molecules (Green (1975) Adv. Protein Chem., 29: 85-133). The avidin gene cDNA has been cloned (Gope et al. (1987) Nucleic Acids Res., 15: 3595-3606), and avidin can be produced in industrial-scale quantities using recombinant DNA technology.

Other transportable molecules are known to those of skill and can be routinely incorporated into the reagents of this invention.

In addition to the use of cognate ligands as targeting ligands, this invention contemplates the use of derivatives or mimetic of such ligands. Such derivatives or mimetics can be produced using standard methods well known to those of skill in the art. For example, routine conservative or semi-conservative substitutions (e.g. E for D) can be made of the existing amino acids.

Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15: 29; Veber and Freidinger (1985) TINS p.392; and Evans et al. (1987) J. Med. Chem. 30: 1229) and are usually developed with the aid of computerized molecular modeling. Peptide mimetics that are structurally similar to therapeutically useful peptides may be used to produce an equivalent therapeutic or prophylactic effect.

Peptidomimetics can be structurally similar to a paradigm polypeptide (i.e., transferrin described herein), but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, —CH₂SO—, etc. by methods known in the art and further described in the following references: Spatola (1983) p. 267 in Chemistry and Biochemistry of Amino Acids, Peptides, and Proteins, B. Weinstein, eds., Marcel Dekker, New York; Spatola (1983) Vega Data 1(3) Peptide Backbone Modifications. (general review); Morley (1980) Trends Pharm Sci pp. 463-468 (general review); Hudson et al. (1979) Int J Pept Prot Res 14:177-185 (—CH₂NH—, CH₂CH₂—); Spatola et al. (1986) Life Sci 38:1243-1249 (—CH₂—S); Hann, (1982) J Chem Soc Perkin Trans 1 307-314 (—CH—CH—, cis and trans); Almquist et al. (1980) J Med Chem. 23:1392-1398 (—COCH₂—); Jennings-White et al. (1982) Tetrahedron Lett. 23:2533 (—COCH₂—); Szelke, M. et al., European Appln. EP 45665 (1982) CA: 97:39405 (1982) (—CH(OH)CH2—); Holladay et al. (1983) Tetrahedron Lett 24:4401-4404 (—C(OH)CH₂—); and Hruby (1982) Life Sci., 31:189-199 (—CH₂—S—)).

A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), reduced antigenicity, and others.

In addition, circularly permutations of the peptides described herein or constrained peptides (including cyclized peptides) comprising a consensus sequence or a substantially identical consensus sequence variation may be generated by methods known in the art (Rizo and Gierasch (1992) Ann. Rev. Biochem. 61: 387); for example, by adding internal cysteine residues capable of forming intramolecular disulfide bridges which cyclize the peptide.

The peptide ligands used in this invention are chemically synthesized using standard chemical peptide synthesis techniques or are recombinantly expressed. In preferred embodiments, the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well know to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

B) Antibodies.

In certain embodiments, the targeting ligand is an antibody that specifically binds to a receptor that is found on a cell comprising the blood brain barrier, and thereby mimics the action of the endogenous peptide ligand, and such antibodies are called peptidomimetic antibodies. Such are receptors are well known to those of skill in the art (see, e.g., U.S. Pat. Nos. 4,801,575, 6,287,792, etc.) and include, but are not limited to the transferrin receptor and the insulin receptor.

Antibodies to these and other suitable receptors are readily available from either commercial sources or can be produced using routine methods well known to those of skill in the art. Thus, for example, the OX26 monoclonal antibody (Pardridge (1997) J. Cereb. Blood Flow Metabol., 17: 713-731) is suitable. This antibody is directed against the rat transferrin receptor (TfR) and undergoes receptor-mediated transcytosis through the blood brain barrier in vivo.

Other antibodies can also be used. For example, for use in humans, the rat TfR antibody can be replaced with an antibody directed against the human insulin receptor (see, Pardridge et al. (1995) Pharm. 12: 807-815).

Chimeric, humanized, or human intact antibodies, or fragments, or single chain human antibodies can also be routinely prepared. Thus, for example, fully human single chain antibodies can be produced.

While, in certain embodiments, either polyclonal or monoclonal antibodies may be used in the reagents of this invention, monoclonal antibodies are preferred. Polyclonal antibodies are typically raised by multiple injections (e.g. subcutaneous or intramuscular injections) of the antigen in question (e.g. a receptor protein) into a suitable non-human mammal.

If desired, the immunizing peptide may be coupled to a carrier protein by conjugation using techniques which are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit). The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, e.g., Coligan, et al. (1991) Unit 9, Current Protocols in Immunology, Wiley Interscience).

Preferred antibodies used in this invention are monoclonal antibodies (“mAb's”). For preparation of monoclonal antibodies, immunization of a mouse or rat is preferred. The term “antibody” as used in this invention includes intact molecules as well as fragments thereof, such as, Fab and F(ab )²′ which are capable of binding an epitopic determinant.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines.

Confirmation of the desired specificity among mAb's can be accomplished using relatively routine screening techniques (such as the enzyme-linked immunosorbent assay, or “ELISA”) to determine the elementary reaction pattern of the mAb of interest.

Antibody fragments or various single chain antibodies (scFv or others), can also be produced/selected using phage display technology. The ability to express antibody fragments on the surface of viruses that infect bacteria (bacteriophage or phage) makes it possible to isolate a single binding antibody fragment from a library of greater than 10¹⁰ nonbinding clones. To express antibody fragments on the surface of phage (phage display), an antibody fragment gene is inserted into the gene encoding a phage surface protein (pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628).

It will also be recognized that numerous antibodies are commercially available and that antibodies can be prepared by any of a number of commercial services (e.g., Berkeley antibody laboratories, Bethyl Laboratories, Anawa, Eurogenetec, etc.).

II. Nucleic Acid.

The nucleic acid sequence is selected such that it is capable of hybridizing with the target nucleic acid that it is desired to detect or quantify. The nucleic acid can be full length or less than the full length of the target nucleic acid (e.g. gene or RNA). In preferred embodiments, short nucleic acids are empirically tested for specificity. Preferred nucleic acids are sufficiently long so as to specifically hybridize with the target nucleic acid(s) under stringent conditions. The preferred size range is from about 10 bases to the length of the target, preferably from about 15 or 20 bases to the length of the target, more preferably from about 30 bases to the length of the target, and most preferably from about 40 bases to the length of the target. In a most preferred embodiment, the nucleic acid sequence is selected to be complementary to its target sequence. Where the target sequence is an mRNA, in preferred embodiments, the nucleic acid comprising the reagent is an “antisense” nucleic acid.

Nucleic acids for use in this invention include polynucleotides formed from naturally occurring bases and/or cyclofuranosyl groups joined by native phosphodiester bonds. The nucleic acids may also include non-naturally occurring portions. Thus, oligonucleotides may have altered sugar moieties or inter-sugar linkages. Exemplary among these are the phosphorothioate and other sulfur containing species that are known for use in the art.

In accordance with some preferred embodiments, at least one of the phosphodiester bonds of the oligonucleotide has been substituted with a structure which functions to enhance the ability of the compositions to penetrate into the region of cells where the RNA whose activity is to be detected is located. It is preferred that such substitutions comprise phosphorothioate bonds, methyl phosphonate bonds, short chain alkyl cycloalkyl structures, and/or phenoxazine. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides can also include species that include at least some modified base forms. Thus, purines and pyrimidines other than those normally found in nature may be so employed. Similarly, modifications on the furanosyl portions of the nucleotide subunits can also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Methods of preparing such nucleic acid, including peptide nucleic acids are well known to those of skill in the art, e.g. as taught in the references identified above.

III. Detectable Label.

The imaging reagents of this invention are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ¹¹¹In, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

In particularly preferred embodiments, the labels include labels detectable by non-invasive imaging techniques. Such labels include, but are not limited to radioactive labels, radioopaque labels, spin labels, magnetic or paramagnetic labels, and the like. In most preferred embodiments, the labels are radio labels (e.g. for detection via SPECT or PET), or magnetic labels (e.g. galdolinium), for detection by MRI.

It will be recognized that labels are not to be limited to single species organic molecules, but include inorganic molecules, multi-molecular mixtures of organic and/or inorganic molecules, crystals, heteropolymers, and the like.

The labels can be attached to the nucleic acid and/or to the targeting ligand directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

In preferred embodiments, particularly where the nucleic acid is a peptide nucleic acid, a label is attached by coupling of a labeled (e.g. radio labeled) amino acid to the peptide nucleic acid. Thus, for example, in one instance, a ¹²⁵Tyrosine is attached to the peptide nucleic acid, while in another instance, a ¹¹¹In lysine is attached to the peptide nucleic acid.

IV. Assembly of the Reagent.

The reagents of this invention can be assembled according to standard methods well known to those of skill in the art. The nucleic acid (e.g. peptide nucleic acid) component can be assembled using well-known solid phase synthesis chemistries. These chemistries permit ready coupling of an affinity tag (e.g. biotin, avidin, streptavidin, etc.) either directly or through a linker.

Similarly, the targeting ligand can be chemically synthesized or recombinantly expressed. Solid-phase peptide synthesis chemistries are also well known to those of skill in the art (see, e.g., Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.).

The targeting ligand and the nucleic acid are joined by any of a number of means well known to those of skill in the art. In particularly preferred embodiments, they are joined directly, through a linker, or by means of an affinity tag. One of skill will appreciate that the targeting molecule and nucleic acid may be joined together in any order. The targeting molecule may also be joined to an internal region of the nucleic acid, or conversely, the effector molecule may be joined to an internal location of the targeting ligand, as long as the attachment does not interfere with the respective activities of the molecules.

Where an affinity tag is utilized to couple the targeting ligand to the nucleic acid, the components are simply brought together and the moieties comprising the affinity tag typically form a covalent linkage. It is noted however, that the linkage need not be covalent. Thus, for example, where the affinity tag comprises an antibody the coupling can be mediated by a combination of ionic bonds, hydrophobic interactions, and the like.

The terms “epitope tag” or “affinity tag” are used interchangeably herein, and refer to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, His₆ bound by Ni—NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO: 1) epitope, c-myc antibodies (available from Sigma, St. Louis), the HNK-1 carbohydrate epitope, the HA epitope, the HSV epitope, the His₄, His₅, and His₆ epitopes that are recognized by the His epitope specific antibodies (see, e.g., Qiagen), and the like. In addition, vectors for epitope tagging proteins are commercially available. Thus, for example, the pCMV-Tag1 vector is an epitope tagging vector designed for gene expression in mammalian cells. A target gene inserted into the pCMV-Tag1 vector can be tagged with the FLAG® epitope (N-terminal, C-terminal or internal tagging), the c-myc epitope (C-terminal) or both the FLAG (N-terminal) and c-myc (C-terminal) epitopes.

In certain embodiments, the targeting ligand and the nucleic acid are chemically conjugated to each other. Means of chemically conjugating molecules are well known to those of skill.

Many procedures are known for attaching a targeting molecule (e.g. an antibody) to another moiety (e.g. to a nucleic acid). Polypeptides typically contain variety of functional groups; e.g., carboxylic acid (COOH) or free amine (—NH₂) groups, that are available for reaction with a suitable functional group on a nucleic acid or on a linker attached to the nucleic acid.

Alternatively, the targeting ligand and/or the nucleic acid may be derivatized to expose or attach additional reactive functional groups. The derivatization may involve attachment of any of a number of linker molecules such as those available from Pierce Chemical Company, Rockford Ill.

A “linker”, as used herein, is a molecule that is used to join the targeting ligand to the nucleic acid. The linker is preferably capable of forming covalent bonds to both the targeting ligand and to the nucleic acid. Suitable linkers are well known to those of skill in the art and include, but are not limited to, straight or branched-chain carbon linkers, heterocyclic carbon linkers, or peptide linkers. Where the targeting molecule is an antibody and the nucleic acid is a peptide nucleic acid, the linkers may conveniently attached through the terminal carboxyl or amino groups. In the case of the polypeptide, the linker can be joined to the constituent amino acids through their side groups (e.g., through a disulfide linkage to cysteine).

A bifunctional linker having one functional group reactive with a group on the nucleic acid, and another group reactive with a group on the targeting ligand (e.g. antibody), may be used to form the desired conjugate. Alternatively, derivatization may involve chemical treatment of the targeting molecule, e.g., glycol cleavage of the sugar moiety of a the glycoprotein antibody with periodate to generate free aldehyde groups. The free aldehyde groups on the antibody may be reacted with free amine or hydrazine groups on an agent to bind the agent thereto. (See U.S. Pat. No. 4,671,958). Procedures for generation of free sulfhydryl groups on polypeptides, such as antibodies or antibody fragments, are also known (See U.S. Pat. No. 4,659,839).

Many procedures and linker molecules for attachment of various compounds including radionuclide metal chelates, toxins and drugs to proteins such as antibodies are known. See, for example, European Patent Application No. 188,256; U.S. Pat. Nos. 4,671,958, 4,659,839, 4,414,148, 4,699,784; 4,680,338; 4,569,789; and 4,589,071; and Borlinghaus et al. Cancer Res. 47: 4071-4075 (1987). In particular, production of various immunotoxins is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982), Waldmann, Science, 252: 1657 (1991), U.S. Pat. Nos. 4,545,985 and 4,894,443.

In particularly preferred embodiments, the targeting ligand is a receptor-specific antibody (e.g. monoclonal Ab, single-chain Ab, etc.) or ligand attached to the antisense nucleic acid (e.g. a peptide nucleic acid) by use of avidin/biotin technology (see, e.g., U.S Pat. No. 6,287,792, EP 413731, EP 276278, WO 8910134, WO 8800834, and the like). In this approach, biotin is placed at the amino terminus of the peptide nucleic acid (PNA) during the solid phase synthesis of the PNA. Streptavidin (SA) with binds the biotin with extremely high avidity (Green (1975) Adv. Protein Chem., 29: 85-133) is joined to the targeting ligand either chemically typically using a linker (e.g. via a stable thioether linkage) or using genetic engineering and production of antibody-streptavidin or antibody-avidin fusion proteins.

V. Inhibition of Gene Expression.

Having demonstrated herein, that it is possible to deliver antisense molecules for imaging purposes across both the blood brain barrier and the cell membrane in sufficient quantity to monitor levels of gene expression, one of skill will appreciate that the same methods can be utilized to specifically modulate or inactivate expression of one or more target genes. In a preferred embodiment, such methods will involve substituting the nucleic acid labeled with a detectable label with a nucleic acid that inactivates or inhibits expression of a target gene. Such nucleic acids are known to those of skill in the art and include, but are not limited to antisense molecules, catalytic nucleic acids (e.g. ribozymes, catalytic DNAs), and inhibitory RNA (RNAi).

In general the methods are performed in the same manner as the in vivo brain imaging described above. A construct comprising a targeting ligand attached to a nucleic acid that is an inhibitory nucleic acid, preferably complementary to the mRNA of the gene or genes it is desired to inhibit is administered to the organism in a concentration sufficient to reduce or fully inhibit transcription of the target gene in a brain cell.

Such methods can be used in a wide variety of contexts. For example, oncogenes activated in various brain tumors can be selectively inhibited, receptors overexpressed in various neuropathologies (e.g. schizophrenia) can be selectively downregulated, and so forth.

A) Antisense Approaches.

Target gene expression can be downregulated or entirely inhibited by the use of antisense molecules. A preferred antisense sequence or antisense nucleic acid is a nucleic acid that is complementary to the coding mRNA of the target gene or a subsequence thereof. Binding of the antisense molecule to the target mRNA interferes with normal translation of the polypeptide encoded by the mRNA. Such antisense oligonucleotides can be essentially identical to the antisense nucleic acids used for the labeling reagents, but, in this instance, need not be labeled with a detectable label.

B) Catalytic RNAs and DNAs

1) Ribozymes.

In another approach, expression of one or more target genes can be can be inhibited by the use of ribozymes as the “nucleic acid” comprising the constructs described herein. The ribozyme is capable of inhibiting/degrading a target mRNA when it is transported across the blood brain barrier and into a brain cell. As used herein, “ribozymes” include RNA molecules that contain anti-sense sequences for specific recognition, and an RNA-cleaving enzymatic activity. The catalytic strand cleaves a specific site in a target mRNA, preferably at greater than stoichiometric concentration.

Because both hammerhead and hairpin ribozymes are catalytic molecules having antisense and endoribonucleotidase activity, ribozyme technology has emerged as a powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Such ribozymes can be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically bind to and cleave a target nucleic acids.

The ribozymes used in the constructs of this invention can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio or other analogs to render the ribozyme resistant to endonuclease activity.

2) Catalytic DNA

In another embodiments, the nucleic acid component of the constructs of this invention can comprise a catalytic DNA that is capable of inhibiting/degrading a target mRNA when it is transported across the blood brain barrier and into a brain cell. In a manner analogous to ribozymes, DNAs are also capable of demonstrating catalytic (e.g. nuclease) activity. Highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to any particular mRNA like a ribozyme.

C) Knockout Constructs.

In another approach, the nucleic acid comprising the reagents of this invention can be substituted with a “knockout construct” capable of binding to and inserting itself in a target gene or gene promoter and thereby disrupting expression of that gene. Such disruption can be specifically directed to a target gene by homologous recombination where a “knockout construct” contains flanking sequences complementary to the domain to which the construct is targeted. Insertion of the knockout construct into a the target gene or promoter results in disruption of expression of that gene.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395). The use of homologous recombination to alter expression of endogenous genes is also described in detail in U.S. Pat. No. 5,272,071, WO 91/09955, WO 93/09222, WO 96/29411, WO 95/31560, and WO 91/12650.

VI. Administration of Imaging or Modulating Reagents.

The imaging or expression modulating reagents of this invention are introduced into the body by any conventional procedure including, but not limited to oral, nasal, topical, transdermal, rectal, and parenteral routes. Preferred parenteral routes include, but are not limited to, subcutaneous, intradermal, intravenous, intramuscular and intraperitoneal routes. Particularly preferred routes include oral administration and intranasal administration.

The reagents are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

Preferably the reagents are combined with a compatible pharmaceutical carrier and injected parenterally or if desired combined with a suitable carrier and administered intranasally in accordance with the well-known conventional procedures used for intranasal administration of insulin. Suitable carrier solutions include those commonly used in injectable or nasal-inhaled hormone preparations such as sterile saline at a pH of around 5 which includes common bacteriostatic agents.

The concentration of the imaging reagent in the carrier will vary depending upon the imaging reagent. Preferably, levels of the reagent in the carrier should be between about 0.0001 weight percent to about 1 weight percent, more preferably about 0.001 weight percent to about 0.1 weight percent. As a general rule, the dosage levels are optimized according to standard methods well known to those of skill in the art.

VII. Kits Comprising Imaging Reagents.

In still another embodiment, this invention provides kits for practice of the methods described herein. In certain embodiments, the kits comprise one or more imaging reagents or expression modulating reagents described herein. The reagents can be provided with the targeting ligand already conjugated to the nucleic acid, or the targeting ligand and nucleic acid can be provided in separate containers. In the latter embodiment, a plurality different nucleic acids can be provided along with one or a variety of targeting ligands, and the desired targeting ligand/nucleic acid combination can be determined by the end user.

The reagent or reagent components can be provided in solution/suspension (e.g. a buffer, excipient, etc.), or in a lyophilized/dried form.

The kits can optionally include any reagents and/or apparatus to facilitate practice of the methods described herein. Such reagents include, but are not limited to buffers, instrumentation, syringes, and the like.

In addition, the kits may include instructional materials containing directions (i.e., protocols) for the practice of the methods of this invention. Preferred instructional materials provide protocols utilizing the kit contents for screening for a gene or gene expression in the brain or brain tissue of an organism. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

Antisense Imaging of Gene Expression in the Brain In Vivo

Antisense radiopharmaceuticals could be used to image gene expression in the brain in vivo, should these polar molecules be made transportable through the blood-brain barrier (BBB). This example describes an antisense imaging reagent comprised of an iodinated peptide nucleic acid (PNA) conjugated to a monoclonal antibody to the rat transferrin receptor using avidin-biotin technology. The PNA was a 16-mer antisense to the sequence around the methionine initiation codon of the luciferase mRNA. C6 rat glioma cells were permanently transfected with a luciferase expression plasmid and C6 experimental brain tumors were developed in adult rats.

The expression of the luciferase transgene in the tumors in vivo was confirmed by the measurement of luciferase enzyme activity in the tumor extract. The [¹²⁵I]-PNA conjugates was injected intravenously in anesthetized animals with brain tumors and sacrificed 2 hours later for frozen sectioning of brain and film autoradiography. No image of the luciferase gene expression was obtained following the administration of either the unconjugated anti-luciferase PNA, or a PNA conjugate that was antisense to the mRNA of a viral transcript. In contrast, tumors were imaged in all rats administered the [¹²⁵I]-PNA that was antisense to the luciferase sequence and was conjugated to the targeting antibody. This example demonstrates that gene expression in the brain n vivo can be imaged with antisense radiopharmaceuticals that are conjugated to a brain drug targeting system.

Introduction.

The availability of the human genome sequence will accelerate the pace of the discovery of pathologic genes that cause cancer or chronic disease in brain and other organs, and in vivo gene imaging technology is needed. Gene expression is imaged in vitro with antisense technology based on the complementary hybridization of an antisense agent with a target mRNA sequence. However, the extension of antisense technology to imaging gene expression in vivo is limited by several factors including rapid metabolism in vivo, toxicity, and poor transport of antisense agents across biological membranes (1). Antisense imaging of gene expression in the brain is particularly difficult because of the presence of the blood-brain barrier (BBB).

Potential antisense imaging agents include phosphodiester (PO) oligodeoxynucleotides (ODN), phosphorothioate (PS)—ODNs, or peptide nucleic acids (PNA). PO—ODNs have been radiolabeled as antisense imaging agents, but these molecules are rapidly degraded in vivo by exonucleases and endonucleases(Kang et al. (1995) Drug Metab. Dispos. 23: 55-59). PS-ODNs are more metabolically stable, but these agents are neurotoxic (Whitesell et al. (1993) Proc. Natl. Acad. Sci., USA, 90: 46654669; Wojcik et al. (1996) J. Pharmacol. Exp. Ther. 278: 404410), probably because of the avidity of PS-ODNs for binding multiple cellular proteins (Rockwell et al. (1997) Proc. Natl. Acad. Sci., USA, 94, 6523-6528), and are strongly bound by plasma proteins (Wu et al. (1996) J. Pharmacol. Exp. Ther. 276: 206-21 1). In addition, PS-ODNs activate RNase H (Crooke (1993) FASEB J. 7: 533-539). Formation of the duplex between the PS-ODN antisense radio pharmaceutical and the target mRNA would lead to degradation of the target transcript, which is not desired in a diagnostic modality. The PNAs have a polyamide backbone (Nielsen et al. (1994) Bioconjugate Chem. 5: 3-7) and are not susceptible to degradation by nucleases(Demidov et al. (1994) Biochem. Pharmacol. 48: 1310-1313). PNAs do not activate RNase H and would appear to be an ideal antisense imaging agent, particularly because the melting point of nucleic acid duplexes formed by PNAs is much higher than with PO or PS-ODNs (Nielsen et al. (1994) Bioconjugate Chem. 5: 3-7). However, PNAs do not cross cell membranes in general and do not cross the BBB (Pardridge et al. (1995) Proc. Natl. Acad. Sci., USA, 92: 5592-5596). Therefore, the successful imaging of gene expression in vivo will require the development of a brain drug targeting technology that can be applied to antisense radiopharmaceuticals.

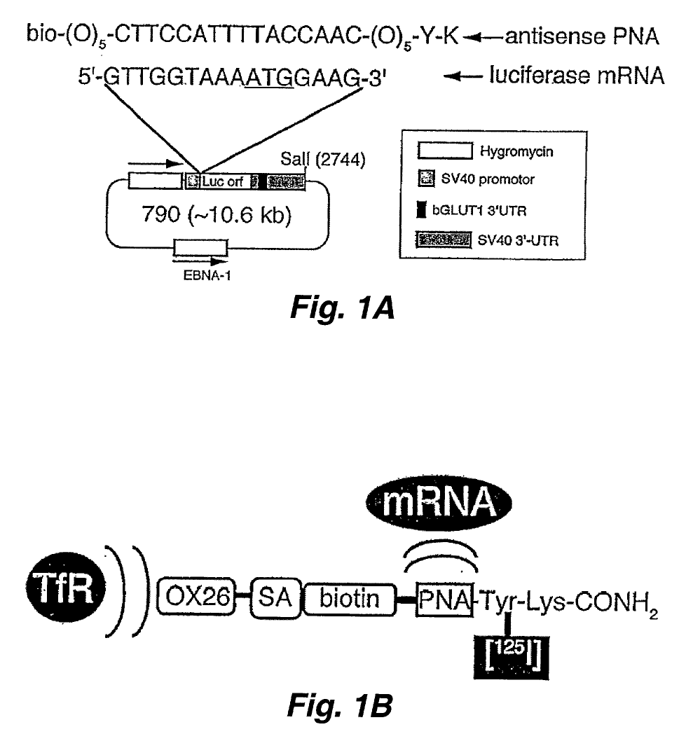

Antisense agents such as PNAs can be made transportable through the BBB with the use of the chimeric peptide technology, as described previously (Id.). In this approach, the drug that is normally not transported through the BBB is biotinylated and then bound to a conjugate of streptavidin (SA) and a brain drug-targeting vector (Pardridge (1997) J. Cereb. Blood Flow Metab. 17: 713-731). The latter is a ligand such as a peptide or peptidomimetic mAb that undergoes receptor-mediated transcytosis (RMT) through the BBB in vivo by virtue of binding to one of several endogenous peptide receptor systems on the brain capillary endothelium, which forms the BBB in vivo. Transferrin or transferrin receptor (TfR) peptidomimetic mAbs, such as the mouse OX26 mAb to the rat TfR, undergo RMT through the BBB in rats in vivo (Id.). In the present example, the conjugate of the OX26 mAb and SA is used and is designated OX26/SA or SA-OX26. The antisense imaging agent is a PNA, which hybridizes to the region around the methionine initiation codon of the luciferase mRNA. C6 rat glioma cells were permanently transfected with a luciferase expression plasmid (FIG. 1A), and the luciferase transgene was expressed in C6 experimental brain tumors in adult rats. The specific expression of the luciferase transgene in the brain tumors was imaged after the i.v. injection of the imaging agent in rats with brain tumors expressing the luciferase gene in vivo.

Experimental Procedures

Conjugate Synthesis.

The sequence of the antiluciferase PNA is shown in FIG. 1A and was synthesized by Applied Biosystems and contained a biotin at the amino terminus followed by 5 linkers, followed by the 16 mer nucleic acid sequence, followed by another 5 linkers, followed by a tyrosine and lysine residue and an amidated carboxy terminus. Each of the five linkers is comprised of NH(CH₂)₂O(CH₂)₂OCH₂CO, which are incorporated in the PNA synthesis by the manufacturer. The calculated molecular mass of the PNA was 6,193 kDa, and the observed molecular mass of the PNA was 6,193 kDa, as determined by mass spectrometry. A control PNA that should not hybridize to any transcripts in brain was prepared with a sequence antisense to the rev gene of the HIV. The antirev PNA had the following nucleic acid sequence: CTCCGCTTCTTCCTGCCA (SEQ ID NO: 4), and has been described previously (Pardridge et al. (1995) Proc. Natl. Acad. Sci., USA, 92: 5592-5596). Either PNA was radioiodinated with 125-iodine and chloramine T, as described previously (Id.), to a specific activity of 75-90 μCi/μg and a trichloroacetic acid (TCA) precipitability of 95-98%. The conjugate of the OX26 mAb and recombinant SA was prepared as previously described by using a stable thioether linkage (Id.).

Luciferase Expression Plasmid.

C6 glioma cells were stably transfected with the luciferase gene by using clone 790, which has been described previously (Boado and Pardridge (1998) Mol. Brain Res. 59: 109-113), and these transfected cells are designated C6-790. Clone 790 contains a 200 bp fragment of the 3′-untranslated region (UTR) of the Glutl glucose transporter mRNA, corresponding to nucleotides 2100-2300 of the GLUT1 mRNA sequence, and this was inserted within the luciferase mRNA 3′-UTR. The insertion of the GLUT1 3′-UTR element into the SV40 3′-UTR maximizes luciferase gene expression in C6 glioma cells by stabilizing the mRNA (Id.).

Experimental Brain Tumors.

The C6-790 cells were implanted in the caudate-putamen nucleus of male CD Fischer 344 rats (Harlan Breeders, Indianapolis, Ind.), weighing 250-275 g under stereotaxic guidance, as described previously (Kurihara et al. (1999) Bioconjugate Chem. 10: 502-511). The animals were examined 14 days later. To confirm expression of the luciferase transgene in the tumor in vivo, tumor extracts were prepared in Promega lysis buffer, and luciferase enzyme activity was measured with luciferin as substrate (Promega) by using a Berthold (Nashua, N.H.) luminometer, as described previously (Boado and Pardridge (1998) Mol. Brain Res. 59: 109-113). A luciferase standard curve was also assayed, and the enzyme activity was expressed as picograms of luciferase equivalent per milligram of tissue protein. The C6-790 cells were grown in tissue culture as described previously (Id.), and these cells were also extracted in Promega lysis buffer for measurement of luciferase activity in the cells grown in tissue culture before implantation in brain. Control experiments were performed with C6 cells described previously (13), and these cells were used to develop brain tumors exactly as described above, except these tumor cells were not transfected with the luciferase transgene.

Autoradiography in Tumor Bearing Rats.

Fourteen days after implantation of C6-790 cells, the rats were anesthetized with ketamine and xylazine for i.v. injection of brain imaging agents, as described previously (Kurihara et al. (1999) Bioconjugate Chem. 10: 502-511). Each rat received 100 μCi of PNA labeled with [¹²⁵I], and 3 groups of rats were studied: Group A rats received anti-luciferase PNA conjugated to OX26/SA; group B received antiluciferase PNA without conjugation to the brain targeting system; and group C received anti-rev PNA conjugated to OX26/SA. In these studies, each rat received 0.2 nmol of PNA and 40 μg (0.2 nmol) of OX26/SA. Each rat was also administered 20 mg of L-tyrosine and 2 mg of sodium iodide i.p. 15 min before the study to block brain uptake of radiolabeled metabolites such as [¹²⁵I]tyrosine or iodide. The animals were decapitated 2 h after i.v. injection of the isotope, the brain was rapidly removed, cut into coronal slabs, immediately frozen in powdered dry ice, and tissue blocks were stored at 270° C. Cryostat sections of 15 mm thickness were prepared on a Bright cryostat and mounted on glass cover slips, which were then exposed to Reflection blue film with intensifying screens (Dupont-NEN). Xray films were exposed at 270° C. for 3 days, followed by development for 1 minute in Kodak developer and fixation for 5 min in Kodak fixer. The x-ray film was scanned in a UMAX flatbed scanner with transparency adapter, cropped in Adobe (Adobe Systems, Mountain View, Calif.) PHOTOSHOP 5.5 on a G4 Power Macintosh, and images were colorized with NIH IMAGE software. After autoradiography, the glass cover slips were stained with Mayer's hematoxylin to visualize the tumor, and these specimens were subsequently scanned and imaged. All brain scans or all autopsy stains were scanned and colorized simultaneously.

Although the autoradiography was performed on frozen sections of brain, the imaging of gene expression was performed in vivo, because the radiolabeled antisense imaging agent was administered in vivo and was not applied to tissue sections in vitro.

Pharmacokinetics and Organ Uptake in Nontumor Bearing Rats.

Either the unconjugated anti-luciferase PNA or the anti-luciferase PNA conjugated to OX26/SA was injected intravenously into ketamine/xylazine anesthetized adult male Sprague-Dawley rats (270-300 g) by using methods described previously (10). The dose of radioactivity in these experiments was 5 μCi/rat of PNA (0.02 nmol) conjugated to 20 μg/rat of OX26/SA (0.1 nmol).

RNase Protection Assay.

The luciferase RNase protection assay demonstrated specific hybridization of the antiluciferase PNA to the target luciferase mRNA despite conjugation to the OX26/SA vector. These methods were identical to those described previously (Pardridge et al. (1995) Proc. Natl. Acad. Sci., USA, 92: 5592-5596). The luciferase RNA was prepared with a luciferase transcription plasmid, designated clone 760, which has been described previously (Tsukamoto et al. (1997) J. Neurochem. 68: 2587-2592). The sense RNA was synthesized with T7 RNA polymerase after linearization of the plasmid with EcoRI. The transcribed RNA was radiolabeled with [³²Pα]ATP, and the correct size of the radiolabeled transcribed sense RNA was determined by agarose/formaldehyde gel electrophoresis followed by film autoradiography. For the RNase protection assay, 0.5 pmol of biotinylated luciferase PNA with or without conjugation to 10 pmol of OX26/SA was added to 10 5 cpm of ³²P-labeled sense luciferase RNA (8 fmol) in 3 μl buffer (50 mM NaCl/5 mM Tris, pH 8/0.5 mM EDTA) and annealed for 30 min at 56° C. Then 15 units of RNase T1 and 0.4 units of RNase A were added to samples in 15 ml of RNase digestion buffer m (Ambion, Austin, Tex.). RNA fragments were analyzed by 7 M urea/20% PAGE and autoradiography, as described previously (Pardridge et al. (1995) Proc. Natl. Acad. Sci., USA, 92: 5592-5596). Labeled RNA and PNA were heat denatured for 2 min at 95° C. and then incubated on ice for 2 min immediately before the experiment or conjugation to OX26/SA.

Uptake and Pulse-Chase Experiments in Cultured C6 and C6790 Cells.

The uptake of ¹²⁵I-labeled antirev or antiluciferase PNA was investigated in C6-790 and C6 cells in the presence or absence of OX26/SA. Cells were grown in 24 well dishes and incubated with 5 μCi/ml (12 nM) [¹²⁵I]PNA with and without OX26/SA (1:1 molar ratio) for 2, 4, 6, or 24 h. The monolayers were then washed 3 times in 2.5 ml cold PBS (10 mM phosphate buffer, pH 7.2/150 mM NaCl) and lysed with 250 μl reporter lysis buffer (Promega), as previously described (Boado and Pardridge (1998) Mol. Brain Res. 59: 109-113). Aliquots of samples (100 μl) and standards (10 μl) were precipitated with TCA, and the percent of medium PNA that was taken up into the TCA-precipitable cellular fraction was measured and expressed either as nanograms PNA per milligram protein or percentage uptake per milligram protein. Twenty-microliter lysate aliquots were also resolved by SDS in a 12% gel. Gels were fixed in 50% MeOH and 10% acetic acid solution for 30 min, incubated in 7% MeOH, 7% acetic acid and 1% glycerol for 5 min, and dried before autoradiography with Kodak BioMax film and intensifying screens. For the pulse-chase study, either control C6 or C6-790 cells were incubated as described above with 10 μCi/ml (24 nM) [¹²⁵I]antiluciferase PNA conjugated to OX26SA for 24 h. The medium was discarded and fresh medium added that contained no additional radiolabeled PNA. TCA-precipitable cellular radioactivity was then measured at 0, 2, 6, or 24 h of incubation.

Results

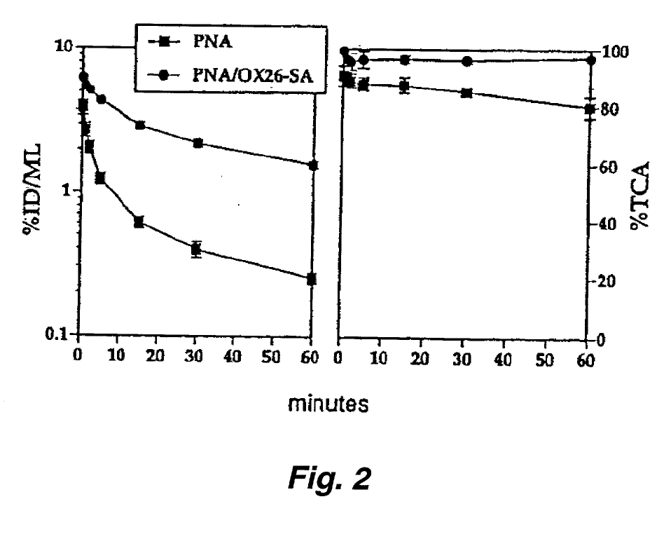

The [¹²⁵I]antiluciferase PNA, with or without conjugation to the OX26/SA drug targeting system, was injected intravenously into adult Sprague-Dawley rats. The profile of plasma radioactivity for the unconjugated PNA or for the PNA conjugate is shown in FIG. 2. The plasma clearance of the unconjugated PNA and of the PNA conjugate was 7.2±0.4 and 1.1±0.1 ml/min/kg, respectively, and the plasma area under the concentration curve (AUC) was inversely related to the plasma clearance. The delayed plasma clearance of the PNA conjugate was paralleled by an increase in metabolic stability as reflected by the high percentage of plasma radioactivity that was precipitable by TCA for at least 60 min after i.v. injection of the PNA conjugate (FIG. 2).

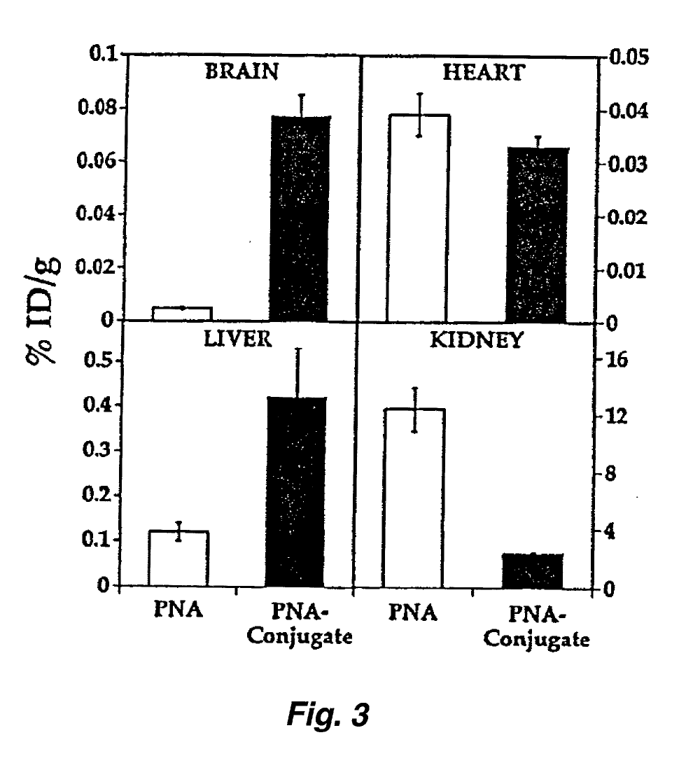

Organ uptake of the radiolabeled PNA or PNA conjugate was measured 60 min after the i.v. injection, and these data are shown in FIG. 3. There was no measurable transport of the unconjugated PNA into brain. However, there was an increase in brain uptake of the PNA after conjugation to the OX26/SA drug-targeting system, and this level of brain uptake, 0.08% of injected dose (ID) per gram of brain, is in excess of the brain uptake of a neuroactive small molecule such as morphine (Pardridge (1997) J. Cereb. Blood Flow Metab. 17: 713-731). There was no specific targeting of the PNA to heart (FIG. 3), because conjugation of the PNA restricts membrane permeability in heart in parallel with an increase in the plasma AUC (FIG. 2), and these have offsetting effects on the percent of ID per gram (Id.). There was increased uptake of the PNA conjugate in liver (FIG. 2) because of the expression of the transferrin receptor on hepatocytes in vivo. There was a decrease in the renal uptake of the PNA conjugate, because conjugation of the PNA to the OX26/SA vector, which has a molecular mass of 200,000 kDa, effectively increases the size of the 6,000 kDa PNA to 206,000 kDa.

The ability of the PNA to hybridize to the target mRNA after conjugation to the OX26/SA drug-targeting system was demonstrated by an RNase A/T1 protection assay. Both the unconjugated and the PNA conjugate hybridized to the luciferase sense RNA and resulted in protection of 16 mer RNA fragments. These results indicated that conjugation of the antiluciferase PNA to the OX26/SA drug-targeting system did not impair the hybridization of the PNA to the target mRNA.

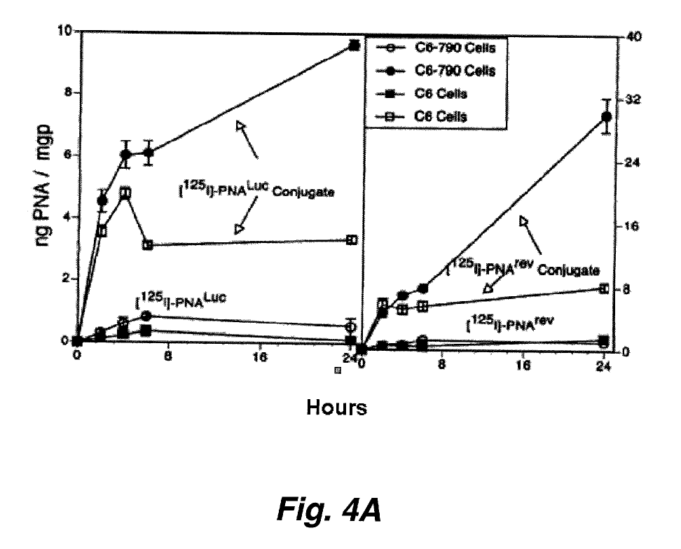

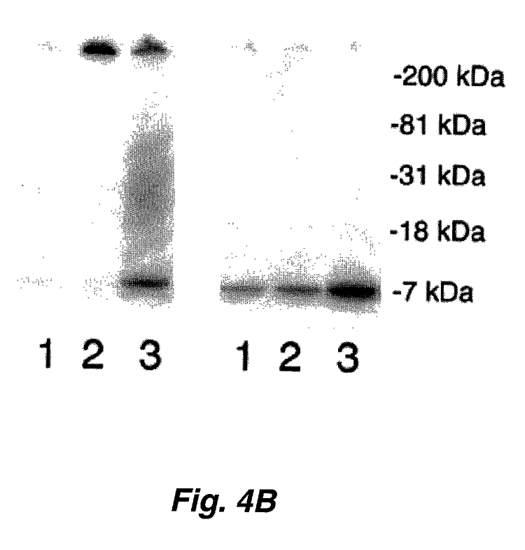

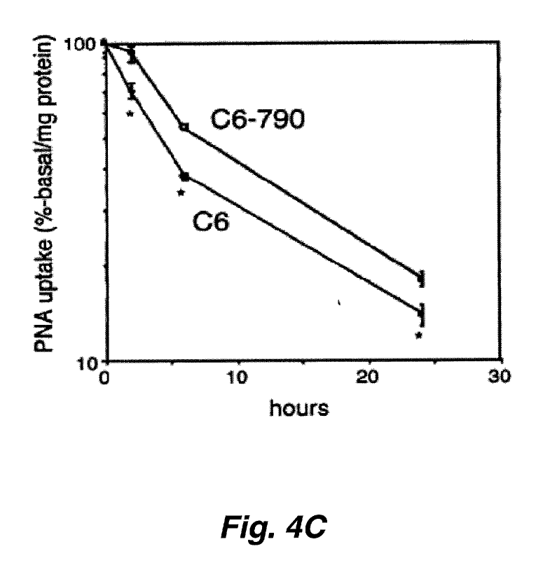

The uptake of either the unconjugated antirev PNA or the unconjugated antiluciferase PNA by either the C6 cells or the C6-790 cells was negligible (FIG. 4A). However, either PNA was taken by these cell lines after conjugation to OX26/SA (FIG. 4A). By 24 h of incubation, the [¹²⁵I]antirev PNA was metabolized, and the [¹²⁵I]tyrosine was recycled into cellular proteins as shown by the SDS-PAGE (FIG. 4B left). However, the [¹²⁵I]antiluciferase PNA was metabolically stable during the 24 h incubation period, as no radioactivity incorporated into cellular proteins was detected (FIG. 4B right). The metabolic stability of the [¹²⁵I]antiluciferase PNA enabled further pulse-chase experiments, and these showed preferential retention of the [¹²⁵I]antiluciferase PNA in the C6-790 cells, compared with the C6 cells lacking the luciferase mRNA (FIG. 4C).

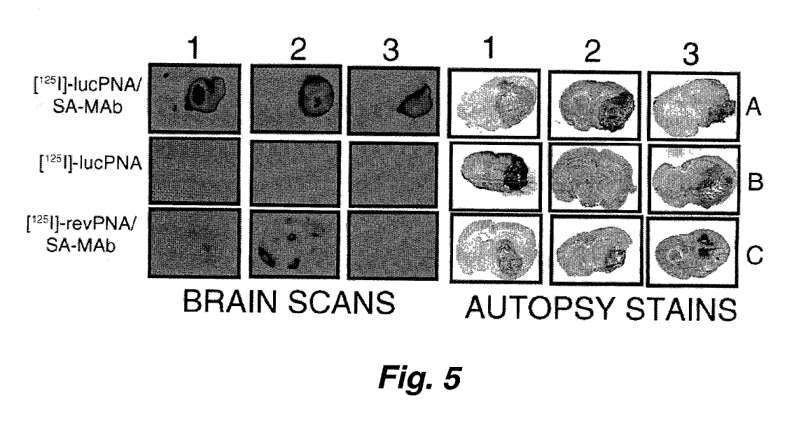

The brain scans and autopsy stains for three different groups of adult Fischer rats bearing the C6-790 gliomas are shown in FIG. 5. The luciferase activity in the tumor extract and in the C6-790 cells in tissue culture was 204 6±66 and 76±2 pg equivalent per milligram of tissue protein, respectively, indicating the luciferase transgene was fully expressed in the experimental tumor in vivo. Group A rats received the radiolabeled antiluciferase PNA conjugated to the OX26/SA drug-targeting system, which is designated SAmAb in FIG. 5. Group B rats received the antiluciferase PNA without conjugation to the drug-targeting system. Group C rats received the antirev antisense PNA that was conjugated to the OX26/SA drug-targeting system. All rats formed medium to large tumors with the exception of rat 2 in group B, as shown by the autopsy stains (FIG. 5). There was no imaging of either normal brain or brain tumor in the group B rats after i.v. injection of the luciferase PNA without conjugation to the drug-targeting system, because the PNA does not cross the BBB in either normal brain or in the tumor. Conversely, there was imaging of luciferase gene expression in the brain tumor in all group A rats after i.v. injection of the luciferase PNA conjugated to the drug-targeting system. The size of the tumor imaged with the antisense radiopharmaceutical was comparable to the size of the tumor shown on the autopsy stain (FIG. 5). In contrast, there was no imaging of the tumors after conjugation of the rev antisense PNA to the drug-targeting system as shown in the group C rats (FIG. 5). In further control experiments, C6 cells not transfected with the luciferase transgene (Kurihara et al. (1999) Bioconjugate Chem. 10: 502-511) were grown as experimental tumors in 10 rats. At 14 days after implantation, 5 rats received 100 μCi each of the unconjugated [¹²⁵I]antiluciferase PNA, and 5 rats received 100 μCi of 125I-labeled antiluciferase PNA conjugated to the OX26/SA drug-targeting system. Brains were removed at 2 h, frozen sections prepared, and film autoradiography performed with the same methods used for the studies shown in FIG. 5. The brain sections were scanned in parallel with the sections from the group A rats (FIG. 5), and no measurable radioactivity was detectable in these control C6 tumors with either the unconjugated or conjugated antiluciferase PNA.

Discussion

These studies are consistent with the following conclusions. First, it is not possible to image gene expression in the brain in vivo with an unconjugated antisense radiopharmaceutical, because these molecules do not cross the BBB in vivo. Second, antisense imaging of gene expression in the brain in vivo is possible if a BBB drug targeting technology is used. The development of an antisense imaging agent for in vivo applications requires the merger of antisense technology and drug-targeting technology.

The antisense imaging agent is comprised of four domains (FIG. 1B). The first domain is the peptidomimetic mAb that targets the TfR, which is expressed on both the BBB and the tumor cell membrane (Kurihara et al. (1999) Bioconjugate Chem. 10: 502-511). Transport through both of these membranes is required because the target of the antisense imaging agent, the luciferase mRNA, is localized in the cytoplasm of the tumor cells. The TfR is expressed on brain cells (Mash et al. (1990) J. Neurochem. 55: 1972-1979) and on C6 glioma cells (Kurihara et al. (1999) Bioconjugate Chem. 10: 502-511), and the data in FIG. 4A show the increased uptake of the PNA conjugate by C6 cells. The data in FIG. 3 show increased transport across the BBB of the PNA after conjugation to the targeting vector. Therefore, the targeting system enables transport of the PNA across both the BBB and the C6 tumor cell membrane. The second part of the imaging agent is the linker domain, comprised of the SA moiety, which is attached to the mAb through a stable thioether linkage, and the biotin moiety, which is incorporated at the amino terminus of the PNA, as shown in FIG. 1B. The third domain of the antisense imaging agent is the radionuclide. At the carboxy terminus of the PNA, there are tyrosine (Y) and lysine (K) residues to enable radiolabeling with either 125-iodine or 111-indium, respectively. In the present study, the PNA was radiolabeled on the tyrosine residue with 125-iodine. The carboxy terminus of the PNA is amidated to enhance resistance to carboxypeptidases. The fourth domain of the imaging agent is the antisense sequence of the PNA which hybridizes to the target mRNA (FIG. 1). The RNase protection assay demonstrates hybridization of the PNA to the target mRNA despite conjugation to the drug-targeting vector.

The experimental model used in these studies is a C6 glioma brain tumor that expresses the luciferase gene in vivo (Results). The C6 cells were permanently transfected with the luciferase gene, and these cells produce high levels of the luciferase mRNA. The abundance of the luciferase mRNA in these cells is comparable to that of the actin mRNA (Boado and Pardridge (1998) Mol. Brain Res. 59: 109-113; Boado et al. (1999) Proc. Natl. Acad. Sci., USA, 96: 12079-12084). This high expression of the luciferase mRNA is caused by the insertion of a cis element derived from the Glut1 glucose transporter mRNA 39 UTR into the luciferase mRNA 3′-UTR. This modification greatly stabilizes the luciferase mRNA and augments the cellular level of the transcript (Boado and Pardridge (1998) Mol. Brain Res. 59: 109-113). Therefore, the gene targeted in the present studies is expressed at high levels, and this expression is exclusive to the tumor cell with no luciferase gene expression in other cells of brain. These factors contribute to the marked differences in imaging the tumor vs. normal brain by using the PNA conjugate (FIG. 5).

The brain scans in FIG. 5 show that the tumor expressing the luciferase transgene is not imaged with a PNA radiopharmaceutical that is not conjugated to a brain drugtargeting system (group B, FIG. 5). These findings corroborate the brain uptake measurements in control rats with the antiluciferase PNA (FIG. 3) and previous studies showing no transport of a PNA across the BBB (Pardridge et al. (1995) Proc. Natl. Acad. Sci., USA, 92: 5592-5596). Antisense molecules are highly charged and form extensive hydrogen bonding in aqueous solution, which restricts the transport across the endothelial plasma membranes forming the BBB in vivo (Pardridge (1997) J. Cereb. Blood Flow Melab. 17: 713-731). Tyler et al. (1999) Proc. Natl. Acad. Sci., USA, 96: 7053-7058 report that unconjugated PNAs do cross the BBB in vivo. In this study, the uptake of the PNA by rat brain was measured with a gel shift analysis of extracts of saline perfused brain. However, this report shows the brain uptake of the PNA is 0.0001% ID/g (Id.), which is a level of brain uptake comparable to that of sucrose, a molecule that traverses the BBB at the lower limit of detection (Pardridge (1997) J. Cereb. Blood Flow Metab. 17: 713-731). In contrast, the brain uptake of the PNA conjugate is 3 logarithm orders of magnitude greater (FIG. 3), and this higher brain uptake enables imaging of gene expression in vivo (FIG. 5).

A radiolabeled PS-ODN has been reported to cross the BBB to enable imaging of gene expression for glial fibrillary acidic protein in experimental brain tumors in rats (Kobori et al. (1999) NeuroReport 10: 2971-2974). However, this study actually uses a drug targeting technology, because the [¹¹C]PS-ODN 25 mer included cholesterol conjugated at the 3′-terminus. The addition of a cholesterol moiety to ODNs increases cellular uptake in tissue culture (de Smidt et al. (1991) Nucleic Acids Res. 19: 4695-4700; Krieg et al. (1993) Proc. Natl. Acad. Sci., USA, 90: 1048-1052). The conjugation of cholesterol to drugs is a “lipidization” drug targeting strategy. The problem with this approach is that the cholesterol adduct is soluble only in organic solvents, and the i.v. administration of these solvents can cause solven-tmediated disruption of the BBB. In studies with the 3′-cholesterol [¹¹C]PS-ODN, the conjugate was solubilized in dichloromethane before i.v. injection (Kobori et al. (1999) NeuroReport 10: 2971-2974). Dichloromethane is a solvent that is neurotoxic (Rebert et al. (1990) Pharmacol. Biochem. Behav. 36: 351-356).

The imaging of gene expression in brain requires the use of an antisense agent with the correct sequence, as the brain tumor was not imaged with an antirev PNA conjugated to SA-OX26 (group C, FIG. 5). The brain uptake of the antirev PNA conjugated to SA-OX26 is higher than the brain uptake of a PNA administered without conjugation to the targeting system (Pardridge et al. (1995) Proc. Natl. Acad. Sci., USA, 92: 5592-5596). However, the differential brain uptake between the conjugated PNA (group C, FIG. 5) and the unconjugated PNA (group B, FIG. 5) is not observed with the film autoradiography, because the brain radioactivity in either case is below the limits of detection. The exposure of the film to the brain sections was limited to 3 days (Experimental Procedures), because this duration was sufficient to image the region of interest, which was the brain tumor. The absence of tumor imaging in the group C rats (FIG. 5) shows that tumor imaging with the targeted antiluciferase PNA is not derived from binding of the anti-TfR mAb to the tumor cells and is not derived from leakiness of the C6 glioma. Similar findings were made with imaging of brain tumors with peptide radiopharmaceuticals conjugated to the SA-OX26 targeting system. In these studies, radiolabeled human epidermal growth factor (EGF) was conjugated to OX26 and administered to rats with C6 experimental tumors. However, no imaging of the tumor was observed, because the C6 cells did not express the EGF receptor (Kurihara et al. (1999) Bioconjugate Chem. 10: 502-511). In another study, the EGF peptide radiopharmaceutical that was conjugated to OX26 was administered to nude rats bearing U87 human glial brain tumors that did overexpress the human EGF receptor (Kurihara and Pardridge (1999) Cancer Res. 54: 6159-6163). In this model, the brain tumor was clearly imaged compared with normal brain (Id.), similar to the result of the present study (FIG. 5). The imaging of structures within the brain with peptide or antisense radiopharmaceuticals requires that two conditions be met. First, the radiopharmaceutical must be enabled to traverse the BBB and/or brain cell membrane so that the radiopharmaceutical can access the target. Second, the region of interest must overexpress the target receptor, in the case of a peptide radiopharmaceutical, or the target mRNA in the case of an antisense radiopharmaceutical. Binding of the radiopharmaceutical to the target receptor or mRNA within the region of interest causes a sequestration of the radioactivity in that region. The selective sequestration of the antiluciferase PNA conjugate by the C6-790 cells is shown in FIG. 4C. The difference between the rate of efflux of the PNA from the C6-790 cells and the control C6 cells in tissue culture is not large. This is because the total number of PNA molecules taken up by the cells in the conjugate form is very high and is greatly in excess of the amount of luciferase mRNA. After 24 h of incubation in cell culture, there is 10 ng PNA per milligram of protein (FIG. 4A). This is equivalent to 1.2×10⁶ PNA molecules per cell, given 10⁶ cells per milligram of protein. Therefore, the number of PNA molecules inside the cell is at least 100 fold greater than the number of luciferase mRNA molecules. However, the ratio of PNA/mRNA molecules is much lower in vivo. Given a brain uptake of 0.08% ID/g (FIG. 3), and assuming 100 mg protein per gram of brain and 10⁶ mg protein per cell, then there are only about 900 PNA molecules per cell in vivo. This number most likely approximates the luciferase mRNA copy number in the tumor cells. The approximation of the number of PNA molecules per cell by the number of target mRNA molecules in vivo enables the selective sequestration of the labeled PNA in the target cell in vivo. This accounts for the high signal-to-noise ratio in the tumor relative to normal brain (FIG. 5, group A).

Imaging gene expression with antisense radiopharmaceuticals requires that the imaging agent traverse three membranes in series: the BBB, the brain-target cell membrane, and the intracellular endosomal membrane. PNAs are able to traverse the endosomal membrane once the PNA is taken up by the cell (Chinnery et al. (1999) Gene Ther. 6: 1919-1928). Moreover, the endosomal membrane may be a more formidable barrier in cultured cells than in vivo. Recent studies have shown that 85 nm pegylated immunoliposomes are able to enter the cytoplasm after transport across the BBB and the neuronal plasma membrane. This was inferred from the finding of active β-galactosidase gene expression in brain after the i.v. injection of this exogenous gene (Shi and Pardridge (2000) Proc. Natl. Acad. Sci., USA, 97, 7567-7572).

The brain drug-targeting technology described in these studies in rats uses peptidomimetic mAbs that bind endogenous BBB peptide receptor systems (Pardridge (1997) J. Cereb. Blood Flow Metab. 17: 713-731). The OX26 mAb and transferrin bind to different sites on the BBB TfR, and very large doses, 190 mg/kg of OX26 mAb, are required to inhibit brain uptake of circulating transferrin (Ueda et al. (1993) J. Neurochem. 60: 106-113). The dose of OX26 mAb used in these imaging studies is 160 μg/kg (Experimental Procedures), which is 3 logarithm orders of magnitude lower than the mAb dose that inhibits endogenous transport (Id). The brain drug-targeting technology used in these experiments in rats could be adapted to the imaging of brain gene expression in humans. In this case, the human insulin receptor (HIR) mAb, which is up to 10-fold more active in primates than the TfR mAb (Pardridge (1997) J. Cereb. Blood Flow Metab. 17: 713-731), would be used as the BBB-targeting agent. The insulin receptor is also widely expressed on brain cells (Zhao et al. (1999) J. Biol. Chem. 274: 34893-34902). A genetically engineered chimeric HIR mAb has been prepared, has the same affinity for the HIR as the original murine HIR mAb (Coloma et al. (2000) Pharm. Res. 17: 266-274), and could be used to target antisense imaging agents across the BBB in humans.

### Example 2

Imaging Gene Expression in the Brain In Vivo in a Transgenic Mouse Model of Human Huntington's Disease With an Antisense Radiopharmaceutical and Drug Targeting Technology

Disease-specific genes of unknown function can be imaged in vivo with anti sense radiopharmaceuticals, providing the trans-cellular transport of these molecules is enabled with drug targeting technology. This example describes the production of 16-mer peptide nucleic acid (PNA) that is antisense around the methionine initiation codon of the huntingtin gene of Huntington's disease (HD).

The PNA is biotinylated, which allows for rapid capture by a conjugate of streptavidin (SA) and the rat 8D3 monoclonal antibody (MAb) to the mouse transferrin receptor (TfR), and the PNA contains a tyrosine residue, to enable radiolabeling with ¹²⁵I. The re-formulated PNA antisense radiopharmaceutical that is conjugated to the 8D3 MAb is designated [¹²⁵I]-PNA/8D3. This form of the PNA is able to access endogenous transferrin (Tf) transport pathways at both the blood-brain barrier (BBB) and the brain cell membrane (BCM) and undergoes both import from blood to brain and export from brain to blood via the TfR.

The ability of the PNA to hybridize to the target human huntingtin RNA, despite conjugation to the MAb, was demonstrated with both cell free translation assays and with RNase protection assays. The [¹²⁵I]-PNA/8D3 conjugate was administered intravenously to either littermate control mice or to R6/2 transgenic mice, which express the exon 1 of the human HD gene, and the mice were sacrificed 6 hours later for frozen sectioning of brain and quantitative autoradiography. The studies demonstrate a 3-fold increase in sequestration of the [¹²⁵I]-PNA/8D3 antisense radiopharmaceutical in the brains of the HD transgenic mice in vivo, consistent with the selective expression of the HD exon 1 mRNA in these animals.

These results support the hypothesis that gene expression in vivo can be quantitated with antisense radiopharmaceuticals, providing these molecules are re-formulated with drug targeting technology. Drug targeting enables access of the antisense agent to endogenous transport pathways, which permits passage across the cellular barriers that separate blood and intracellular compartments of target tissues.

Introduction

The availability of the human genome sequence and the emerging applications of functional genomics will lead to the discovery of many novel genes that are expressed in a disease-specific pattern (Lockhart and Winzeler (2000) Nature., 405: 827-836). Such novel genes have known sequence but unknown function. For example, the majority of genes uniquely expressed in brain cancer are genes of unknown function (Pardridge (2001) Drug Discovery Today., 6: 104-106). One way to image in vivo a gene of unknown function is with the use of antisense radiopharmaceuticals that hybridize to a specific nucleotide sequence within the target mRNA molecule. However, the in vivo applications of antisense radiopharmaceuticals have been limited by the poor trans-cellular transport and organ distribution of these molecules in living organisms (Hnatowich (1999) J. Nucl. Med., 40: 693-703). Phosphodiester (PO)-oligodeoxynucleotides (ODN) are often rapidly degraded in vivo by endo- and exo-nucleases (Tavitian et al. (21998) Nat. Med., 4: 467-471). Phosphorothioate (PS)-ODNs are metabolically stable in vivo, but are often avidly bound by serum proteins (Cossum et al. (1993) J. Pharmacol. Exp. Ther., 267: 1181-1190), which retards uptake into tissues (Wu et al. (1996) J. Pharmacol. Exp. Ther., 276: 206-211). In addition, the highly reactive sulfur atoms in the PS-ODN cause these molecules to non-specifically bind to many cellular proteins (Brown et al. (1994) J. Biol. Chem., 269:26801-26805), which causes non-specific sequestration of the PS-ODN.

A third class of antisense radiopharmaceuticals are peptide nucleic acids (PNA), which are metabolically stable in vivo and are not bound by serum or tissue proteins. However, PNAs are poorly transported across biological membranes, and are typically be physically injected into the intracellular space of a cell in tissue culture in order to hybridize to the target mRNA molecule (Hanvey et al. (1992) Science., 258:1481-1486).

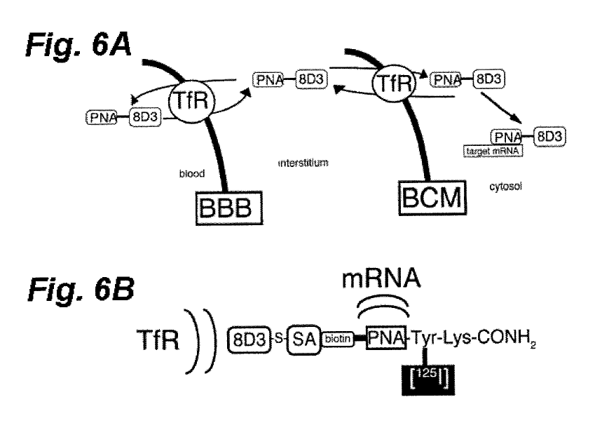

The problem of poor trans-cellular transport of PNA antisense radiopharmaceuticals in vivo is most severe for imaging of transcripts in the brain, because of the blood-brain barrier (BBB). The delivery of a PNA from blood to the intracellular space of brain cells is a “2-barrier” drug targeting problem. The PNA must circumvent both the brain capillary endothelial cell, which forms the BBB in vivo, and then the brain cell membrane (BCM). Both the BBB and the BCM barriers can be traversed with the introduction of drug targeting technology. In this approach, as described herein, trans-cellular transport of the PNA radiopharmaceutical is enabled by conjugation of the PNA to a transport vector. In preferred embodiments, the transport vector is an endogenous peptide or peptidomimetic monoclonal antibody (MAb) that undergoes both receptor-mediated transcytosis across the BBB and receptor-mediated endocytosis across the BCM. The transferrin receptor (TfR) is expressed at both the BBB and the BCM (FIG. 6A).

The OX26 MAb used in studies in rats does not recognize the murine TfR, and is not active in the mouse as a drug-targeting vector (Lee et al. (2000) J. Pharmacol. Exp. Ther., 292:1048-1052). Owing to the use of transgenic mouse models of neurological disease, it would be useful to develop technology that would enable the in vivo imaging of gene expression in the brain in the mouse using sequence specific antisense radiopharmaceuticals. In the present example, the rat 8D3MAb to the mouse TfR (Id.) is used as a brain drug targeting vector to enable imaging of gene expression in vivo in a transgenic mouse model of Huntington's Disease (HD). The R6/2 transgenic mouse model of HD expresses exon 1 of the huntingtin gene, which contains the expanded CAG repeats characteristic of HD (Kirupa et al. (1999) Phil. Trans. R. Soc. Lond., 354: 963-969). Humans without HD have 6-39 CAG repeats in the huntingtin gene whereas patients with HD express 36-180 CAG repeats in this gene. The R6/2 transgenic mouse has one intact copy of human HD exon 1 that contains 114 CAG repeats, and these mice develop neuronal inclusion bodies and behavioral changes as early as 5 weeks after birth (Id.). The HD model was developed for antisense imaging of gene expression in vivo, because prior work showed that sequence specific antisense agents specifically hybridize to target sequences of the huntingtin mRNA (Boado et al. (2000) J. Pharmacol. Exp. Ther., 295: 239-243).

A PNA that specifically hybridizes to exon 1 of the HD gene was synthesized with a carboxyl terminal tyrosine residue to enable radiolabeling with ¹²⁵I (FIG. 6B). The amino terminus of the PNA contains an extended biotin group. The biotin moiety allows for rapid capture by streptavidin (SA), which is covalently conjugated through a stable thiol-ether linkage (—S—) to the 8D3 rat MAb to the mouse TfR (FIG. 6B). The use of avidin-biotin technology allows for the high efficiency coupling of the PNA antisense radiopharmaceutical to the brain drug targeting vector (FIG. 6B).

Materials and Methods

Materials

[¹²⁵I]—Na iodine and autoradiographic [125I] micro-scale 20 μm standard strips were purchased from Amersham-Pharmacia Biotech (Piscataway, N.J.). [³H]-leucine (179 Ci/mmol) and [³²P-α]ATP (800 Ci/mmol) were purchased from Perkin Elmer (Boston, Mass.). The m-maleimidobenzoyl-N-hydroxysuccinimide ester (MBS) and trifluoroacetic acid (TFA) were obtained from Pierce Chemical Co. (Rockford, Ill.). Acetonitrile (HPLC grade) was purchased from Fisher Scientific (Pasadena, Calif.). Recombinant streptavidin (SA), chloramine T and all other reagents were supplied by Sigma-Aldrich, Inc. (St. Louis, Mo.). C18 Sep-Pak extraction cartridges were obtained from Waters Corporation (Milford, Mass.). Male BALB/c mice (25-30 g) were purchased from Harlan Sprague-Dawley (San Diego, Calif.). Huntington's disease exon 1 transgenic mice, male, 7-8 weeks old, 20-25 g [Strain name: B6CBA-TgN(HDexon1)62Gpb], also called R6/2 mice, and littermate control mice were supplied by The Jackson Laboratory (Bar Harbor, Me.). T3-TNT translation system, T3 RNA polymerase and EcoRI were obtained from Promega (Madison, Wis.). Custom oligodeoxynucleotides were purchased from Biosource International (Camarillo, Calif.). RNAse Ti was obtained from Invitrogen (San Diego, Calif.).

Antisense Radiopharmaceutical.

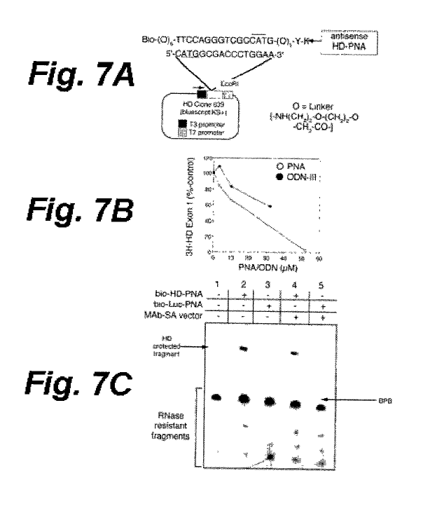

The anti-HD PNA is complementary to nucleotides −1 to +15 of the human HD exon 1 (FIG. 7A). The biotin at the amino terminus is followed by 5 linkers (designated —O—), followed by the 16-mer PNA sequence, followed by another 5 linkers, followed by a tyrosine and lysine residue, and an amidated carboxyl terminus. Each of the 5 linkers is comprised of 2-aminoethoxy-2-ethoxy acetic acid (Applied Biosystems, Foster City, Calif.), which are incorporated during the PNA synthesis. The calculated molecular mass of the PNA was 6316 Daltons and the observed molecular mass of the PNA was 6315 Daltons as determined by mass spectrometry. The HD-PNA was synthesized using an automated synthesis method previously described (Mayfield and Corey (1999) Anal. Biochem., 268: 401-404). A negative control PNA that should not hybridize to the HD transcript was prepared with a sequence antisense to the firefly Luciferase gene as described by Shi et al. (2000) Proc. Natl. Acad. Sci., USA, 97: 14709-14714. The anti-Luciferase PNA had the following nucleic acid sequence: CTTCCATTTTACCAAC (SEQ ID NO: 5), and contained biotin, 5 linkers flanking the nucleotide sequence, tyrosine and lysine in an order identical to HD-PNA (FIG. 7A).

The targeting vector was comprised of a conjugate of recombinant SA and the anti-mouse TfRMAb (FIG. 1B). Two different rat TfRMAb's were evaluated, the 8D3 MAb and the R17-217 MAb (Lee et al. (2000) J. Pharmacol. Exp. Ther., 292:1048-1052). Initial studies showed the conjugate of SA and the 8D3 MAb, designated 8D3-SA, was more active in vivo than the R17-217-SA conjugate, and subsequent studies were performed only with the 8D3-SA conjugate. Owing to the very high affinity of SA for biotin (Green and Avidin (1975) Adv. Prot. Chem., 29: 85-133), there was instantaneous capture of the biotinylated PNA upon mixing with the 8D3-SA vector to form the imaging agent shown in FIG. 6B. The complex of the [¹²⁵I]-PNA bound to the 8D3-SA conjugate constitutes the imaging agent used in the transgenic mouse studies, and is designated the PNA/8D3 conjugate.

Iodination of HD-PNA

Biotinylated HD-PNA (1.8 nmol), [¹²⁵I]—Na iodine (2-4 mCi, 1-2 nmol) and chloramine T (17.7 nmol) were mixed at a total volume of 55 μl of phosphate buffer (pH 7.4) at room temperature for one min. The reaction was stopped by adding sodium metabisulfite (62 nmol), and added to either a C18 Sep-Pak extraction cartridge or to a Sephadex G25 gel filtration column. The Sep-Pak cartridge was washed with 10 ml of 0.1 % TFA and 5 ml of 5% acetonitrile containing 0.1% TFA, and the [¹²⁵I]-HD-PNA was eluted with 5 ml of 40% acetonitrile/0.1% TFA and was stored at 4 ° C. after evaporation of acetonitrile using a Speed Vac Concentrator (Savant Instrument, Inc., Holbrook, N.Y.). Prior to application, the 0.7×28 cm column of Sephadex G-25 was pre-equilibrated with 0.01 M Na₂HPO₄/0.15 M NaCl/pH=7.4/0.05% Tween-20 (PBST), and the sample was eluted with PBST. The final specific activity of the [¹²⁵I]-HD-PNA was 63-120 gCi/g with a trichloroacetic acid (TCA) precipitability of >96%.

In Vivo Pharmacokinetics and Organ Uptake in BALB/c Mice

Adult male BALB/c mice were divided into 5 groups of 3 mice each for the pharmacokinetic study. The mice were anesthetized with an intraperitoneal injection of ketamine (100 mg/kg) and xylazine (2 mg/kg). The injection solution contained 5 μCi of [¹²⁵I]-HD-PNA with or without conjugation to 2 μg of 8D3-SA in 0.01 M PBS/pH 7.4, and a total volume of 50 μl was administered via the jugular vein of each mouse. Groups of 3 mice each were sacrificed at 0.25, 2, 5, 15, and 60 min after the isotope injection and arterial blood was sampled from the aorta. After spinning the blood samples, the collected supernatant (serum) was counted for [¹²⁵I]-radioactivity using a γ-counter (Beckman Instruments, Inc., Fullerton, Calif.). An aliquot of serum was precipitated with cold 10% TCA, and the fraction of total serum radioactivity that was TCA precipitated was determined. The brain and peripheral organs (liver, kidney, heart, lung, and spleen) were removed from each mouse at 60 min after the isotope administration and total [¹²⁵I]-radioactivity in the organ was measured.

For the brain efflux study, BALB/c mice were divided into 4 groups of 3 mice each and 50 μl of PBS/pH 7.4 containing 5 μCi of [¹²⁵I]-HD-PNA with or without 3.2 μg of 8D3-SA conjugate was intravenously injected into the anesthetized mice. Blood was withdrawn from the aorta and animals were decapitated to remove the brain at 1, 2, 4 and 6 hr after injection.

Pharmacokinetic parameters were determined by fitting the serum TCA-precipitable radioactivity data to a biexponential equation with a weighting factor of [1/A(t)]² using a derivative-free nonlinear regression analysis (PARBMDP, Biomedical Computer P-Series, developed at UCLA Health Science Computing Facilities).

A(t)=A₁e^{-K1·t}+A₂e^{-K2·t}

where A(t)=% injected dose (ID)/ml serum at a given time (t).

The organ permeability-surface area (PS) product of either the unconjugated [¹²⁵I]-HD-PNA, or the [¹²⁵I]-HD-PNA conjugated to 8D3/SA, was calculated as follows:

$$PS = \frac{[ {V}_{d} - {V}_{0} ] \cdot {C}_{p} ( t )}{AUC {\vert}_{0}^{t}}$$

where Cp(t) is the terminal serum concentration (% ID/mL), AUC is the area under the serum concentration curve from time 0 to the terminal time (t), V_{d} is the organ volume of distribution, and V₀ is the organ plasma volume, as reported previously (11). The V_{d} of [¹²⁵I]-HD-PNA or [¹²⁵I]-HD-PNA conjugate was calculated from the ratio of CPM/g organ divided by CPM/μl of serum at the terminal time (t) after injection.

The brain delivery of the compound was expressed as percentage of injected dose (ID)/g brain, and was calculated as follows:

% ID/g=[V_{d}−V₀]·Cₚ(t)

Quantitative Autoradiography (QAR)

HD R6/2 transgenic mice and littermate control mice were injected with 150 μl of PBST containing 50 μCi of [¹²⁵I]-HD-PNA and 20 μg of 8D3-SA conjugate (1:1 molar ratio) via the jugular vein under ketamine/xylazine anesthesia, as described above. The animals recovered from anesthesia within 60 min, and were re-anesthetized and sacrificed 6 hr after the isotope administration. The brain of each mouse was rapidly removed, cut into sagittal slabs, immediately frozen in powdered dry ice, and dipped in Tissue-Tek O.C.T. embedding compound. Cryostat sections of frozen brain blocks of 20 μm thickness were prepared on a Mikrome 505HE cryostat (Micron Instruments, Inc., San Diego, Calif.), mounted on glass slides, and dried at room temperature.

For film autoradiography, the slides were exposed to Kodak Biomax MS film for 5 days at −70 ° C. in parallel with 20 μm autoradiographic [¹²⁵I] micro-scale standard strips. The films were then developed for 1 min using a Kodak developer and fixed for 5 min using a Kodak fixer. The films were scanned in a 1200 dpi PowerLookIII UMAX scanner with transparency adapter, and cropped in Adobe Photoshop 5.5 on a G4 Power Macintosh. The integrated density over either the whole brain section or the [¹²⁵I] micro-scale standard was quantified using NIH Image 1.62 software, and normalized by pixel area of the scanned region. The standard curve constructed from [¹²⁵I] micro-scale standards was used to convert the integrated density to brain radioactivity normalized by organ weight (μCi/g).

Translation Arrest

The hybridization of the antisense HD-PNA to the HD transcript was determined using a combination of transcription and translation that mimics in vivo conditions as previously described (Boado et al. (2000) J. Pharmacol. Exp. Ther., 295: 239-243). The cDNA containing the human HD exon 1 was sublconed into the Bluescript KS+plasmid, between either T3 or T7 RNA polymerase promoters, and this plasmid is designated clone 839, as described previously (Id). Transcription/translation of human HD exon 1 clone 839 was performed with 0.2 μg clone 839 plasmid DNA in the presence of T3 RNA polymerase, 5 μCi [³H]-leucine, and 50% v/v rabbit reticulocyte lysate using the T3-TNT translation system (Promega, Madison, Wis.), as described previously (Id). Dose response studies with newly synthesized PNAs were performed and compared with a positive control antisense phosphodiester-ODN, designated ODN-III, as described (Id). Samples were incubated for 30 min at 30 ° C., and incorporation of [³H]-leucine into HD exon 1 protein was analyzed by trichloroacetic acid (TCA) precipitation, and data expressed as percent of control, which lacked any added PNA or ODN. The HD exon 1 insert is 10% of the total plasmid, or about 0.03 μg per 12.5 μL reaction. Since the TNT produces 10-30 RNA copies per plasmid, the final concentration of the HD RNA in the TNT reaction is approximately 1 μM. Therefore, concentrations of 5-50 μM antisense PNA or PO—ODN were used in the TNT assay.

RNase Protection Assay

The HD RNase protection assay (RPA) was used to demonstrate specific hybridization of the anti-HD PNA to the target HD mRNA despite conjugation of the PNA to the TfRMAb-SA vector, as described (Pardridge et al. (1995) Proc. Natl. Acad. Sci., USA, 92:5592-5596). The sense RNA was synthesized with T3 RNA polymerase following linearization of the plasmid with EcoRI. The transcribed RNA was radiolabeled with [³²Pα]ATP to a specific activity of 0.22 μCi/pmol, as previously described (Boado and Pardridge (1994) Bioconj. Chem., 6: 406-410). For the RNAse protection assay, 0.6 pmol of biotinylated HD-PNA or biotinylated luciferase-PNA, with or without conjugation to 6.4 pmol of MAb-SA, was added to 10⁵ CPM of [³²P]-labeled sense HD RNA (0.2 pmol) in 3 μl buffer (80 mM NaCl, 7 mM phosphate buffer, pH=7.5, 0.1% BSA), and annealed for 30 minutes at 42° C. Then 20 units RNAse Ti and 2.5 μg RNAse A were added to the samples in 17 μl of RNAse digestion buffer (0.3 M NaCl, 10 mM TRIS pH=7.5, 4 mM EDTA, 0.02% tRNA, 0.02% BSA) and incubated for 30 min at 37° C. RNA fragments were analyzed by 7 M urea/20% polyacrylamide gel electrophoresis following by autoradiography as described by Pardridge et al. (1995) Proc. Natl. Acad. Sci., USA, 92:5592-5596. Labeled RNA and PNAs were heat denatured for 2 minutes at 95° C. and then incubated on ice for 2 minutes immediately before the experiment or conjugation to MAb-SA.

Results.

The hybridization of the PNA to the HD mRNA is sequence specific (FIG. 7A), and was confirmed with both the transcription/translation assay (FIG. 7B), and the RNase protection assay (RPA) (FIG. 7C). The PNA inhibits the cell free translation of the exon 1 fragment of the HD mRNA in a dose response that is comparable to the dose response inhibition of translation caused by a PO-ODN of the same sequence, and designated PO—ODN-III (FIG. 7B). The hybridization of the PNA to the target HD transcript was verified with the RPA as shown in FIG. 7C (lane 2). The RPA was performed with either the unconjugated PNA (lane 2), which has a molecular weight of 6300 Daltons, or the PNA conjugated to the MAb-SA vector (lane 4), which has a molecular weight of 200,000 Daltons. The presence of a PNA protected RNA fragment following complete nuclease digestion of the HD mRNA is indicative of sequence specific hybridization of the PNA to the target mRNA molecule. The RPA studies in FIG. 7C show that the biotinylated HD PNA specifically hybridizes to the target mRNA, and this hybridization is not altered following conjugation of the PNA to the MAb-SA vector. Conversely, mixing of an anti-luciferase (Luc) PNA with the HD RNA did not protect the RNA from nuclease digestion, either in the unconjugated form (lane 3) or as a conjugate with the MAb-SA vector (lane 5).

Prior to imaging studies in transgenic mice, the pharmacokinetics and organ uptake of the HD PNA were examined in control BALB/c mice for both the unconjugated PNA and the PNA conjugated to 8D3-SA. Blood was sampled from the mice over a 60-minute period following an intravenous injection of either the free or conjugated [¹²⁵I]-PNA. The pharmacokinetic parameters are shown in Table 1.

TABLE 1
Pharmacokinetic parameters. Data are mean ± S.E. computed by
non-linear regression analysis of serum radioactivity measured at
0.25, 2, 5, 15, and 60 min after the intravenous injection of either
the unconjugated [¹²⁵I]-PNA or the [¹²⁵I]-PNA/8D3 conjugate.
A total of 30 mice were used to generate the plasma profiles for
either form of the PNA.

| Parameters | Units | [¹²⁵I]-PNA/8D3 | [¹²⁵I]-PNA |
|---|---|---|---|
| A₁ | % ID/ml | 35 ± 2 | 32 ± 2 |
| A₂ | % ID/ml | 20 ± 1 | 6.3 ± 0.7 |
| K₁ | 1/min | 0.49 ± 0.04 | 0.37 ± 0.04 |
| K₂ | 1/min | 0.0102 ± 0.0008 | 0.0206 ± 0.0014 |
| t_{½}¹ | Min | 1.4 ± 0.1 | 1.9 ± 0.2 |
| t_{½}² | Min | 68 ± 5 | 34 ± 3 |
| AUC (60 min) | % ID · min/ml | 970 ± 47 | 308 ± 36 |
| AUC (∞) | % ID · min/ml | 2048 ± 165 | 399 ± 49 |
| V_{SS} | ml/kg | 176 ± 8 | 374 ± 42 |
| C1 | ml/min/kg | 1.9 ± 0.1 | 9.7 ± 1.3 |

The organ uptake of either the unconjugated PNA or the PNA/8D3 conjugate was assayed at 60 minutes after an intravenous injection of the radiopharmaceutical. The unconjugated PNA was cleared from plasma at a rate of 9.7±1.3 ml/min/kg (Table 1) and the principle organ responsible for clearance of the unconjugated PNA was the kidney (Table 2).

TABLE 2
Organ uptake of the unconjugated PNA or the PNA/8D3 conjugate
at 60 min after intravenous administration in BALB/c mice. Data are
mean ± S.E. (n = 3 mice for each group).

% ID/g

| Organ | [¹²⁵I]-PNA | [¹²⁵I]-PNA/8D3 |
|---|---|---|
| Liver | 0.96 ± 0.02 | 29 ± 2 |
| Kidney | 36 ± 5 | 14 ± 1 |
| Heart | 0.56 ± 0.05 | 1.2 ± 0.1 |
| Lung | 2.1 ± 0.3 | 7.0 ± 0.7 |
| Spleen | 0.64 ± 0.09 | 80 ± 4 |

Conjugation of the PNA to the 8D3-MAb resulted in a nearly 5-fold decrease in the plasma clearance of the radiopharmaceutical (Table 1), owing to a >60% reduction in the renal clearance of the PNA (Table 2). The molecular weight of the PNA/8D3 conjugate, 206,000 Daltons, is 21-fold greater than the molecular weight of the unconjugated PNA (Methods), and the large size of the PNA/8D3 conjugate eliminates glomerular filtration of the PNA. There was minimal uptake of the unconjugated PNA by liver, heart, lung or spleen (Table 2) but the organ uptake of the PNA by TfR-rich organs such as liver or spleen was increased 30- to 100-fold by conjugation to the 8D3 MAb (Table 2). The metabolic stability of the PNA was enhanced following conjugation to the 8D3 MAb, based on measurements of serum radioactivity that was precipitated by TCA. For the unconjugated [¹²⁵I]-PNA, the serum TCA precipitation was 91±1%, 87±2%, and 69±2%, at 5, 15, and 60 min after injection, respectively. For the [¹²⁵I]-PNA/8D3 conjugate, the serum TCA precipitation was 96±1%, 97±1%, 93±1%, 84±1%, and ±3%, at 5, 15, 60, 120, and 360 min after injection, respectively.

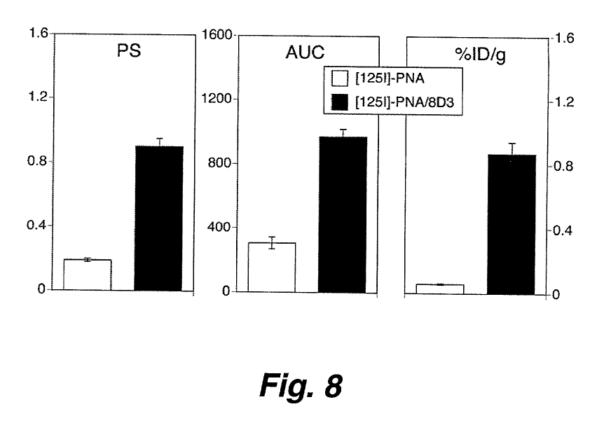

The brain uptake of the unconjugated PNA or the PNA/8D3 conjugate is shown in FIG. 8. The BBB PS product of the unconjugated PNA was negligible, <0.3 μL/min/g, (FIG. 8), which is a very low level of BBB permeability, and comparable to the BBB PS product of sucrose (Samii et al. (1994) Am. J. Physiol., 267: E124-E134), a small molecule that undergoes minimal transport across the BBB in vivo. In contrast, the BBB PS product for the PNA/8D3 conjugate was 1 μL/min/g, which approximates the BBB PS product for the unconjugated 8D3 MAb in control mice (Lee et al. (2000) J. Pharmacol. Exp. Ther., 292:1048-1052). Conjugation of the PNA to the 8D3-SA results in a 3-fold increase in the 60-min plasma AUC (Table 1). Owing to the combined increase in both the BBB PS product and the plasma AUC following conjugation of the PNA to the 8D3-SA, there is a >10-fold increase in the brain uptake of the PNA at 60 minutes after intravenous injection, when expressed as % ID/g (FIG. 8). The uptake of the PNA/8D3 conjugate by the mouse brain approximated 1% ID/g (FIG. 8), which is a level of brain uptake of radioactivity that should yield a measurable signal with the in vivo neuro-imaging studies in the transgenic mice.

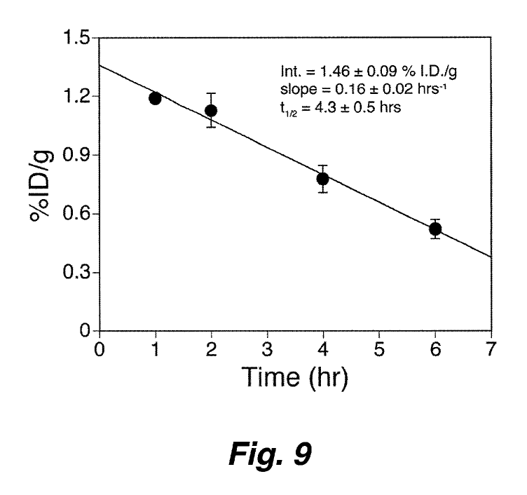

The neuro-imaging of gene expression in vivo with a PNA radiopharmaceutical requires imaging brain at the appropriate time that yields an acceptable signal/noise ratio. The signal is derived from hybridization of the PNA to the target mRNA, and the noise is generated from residual un-bound PNA in brain that has not yet effluxed back to blood. Therefore, following the “import” of the antisense radiopharmaceutical into brain from blood, there is “export” of the antisense radiopharmaceutical back to blood from regions of brain in which there is no specific hybridization of the PNA to the target mRNA (FIG. 6A). Prior to in vivo imaging in the transgenic mice, the export of the PNA/8D3 conjugate from control mouse brain was examined over a 6-hour period. In these studies the PNA/8D3 radiopharmaceutical was administered intravenously and mice were sacrificed at 1, 2, 4, and 6 hours after administration and brain radioactivity was measured. As shown in FIG. 9, there is a mono-exponential decay in brain radioactivity following the initial import of the PNA/8D3 conjugate into the mouse brain. The radioactive conjugate effluxes from mouse brain with a t_{1/2} of 4.3±0.5 hours. Therefore, at 6 hours after intravenous injection, >⅔ of the radioactivity initially imported into the brain has effluxed back to blood. On the basis of these studies, subsequent neuro-imaging in the transgenic mice was performed at 6 hours following intravenous injection of the PNA/8D3 conjugate.

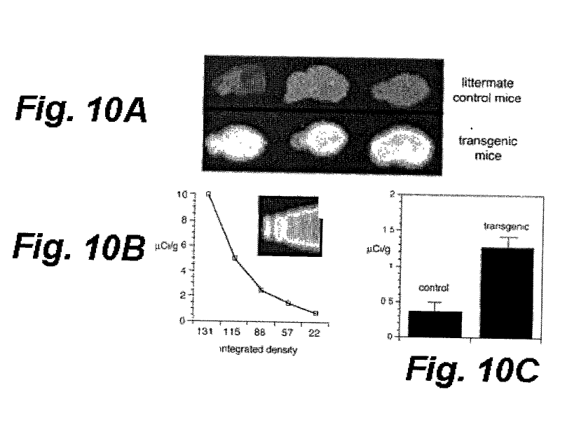

The brain radioactivity of the PNA/8D3 conjugate at 6 hours after intravenous injection is shown for 3 littermate control mice and 3 HD transgenic mice in FIG. 10A. These brain scans were quantitated with the [¹²⁵I] microscale standard strips (FIG. 10B), and the results of the quantitation are shown in panel C of FIG. 5. There is a 3-fold increase in the amount of radioactivity sequestered in the brains of the HD transgenic mice 6 hours after intravenous injection as compared to the littermate control mice (FIG. 10C). The increase in brain radioactivity in the transgenic mice, as compared to the littermate control mice, is also shown by the brain scans (FIG. 10A).

Discussion.

The results of these studies are consistent with the following conclusions. First, the pharmacokinetics and organ uptake of a PNA antisense radiopharmaceutical are profoundly altered by conjugation of the PNA to the MAb targeting vector (Tables 1,2). Second, the brain uptake of the PNA radiopharmaceutical by mouse brain is increased by conjugation to the targeting vector, whereas there is no significant uptake of the unconjugated PNA by mouse brain (FIG. 8). Third, the conjugation of the PNA to the targeting vector enables both the import of the PNA into brain (FIG. 8) followed by the export of the PNA/MAb conjugate back to blood over a 6 hour period (FIG. 9). Fourth, conjugation of the PNA to the targeting vector does not affect hybridization of the PNA to the target HD mRNA, based on either cell free translation or RNase protection assays (FIG. 7). Fifth, the expression of the HD exon 1 gene in the R6/2 HD transgenic mouse can be detected in vivo with the combined use of a sequence specific PNA radiopharmaceutical and brain drug targeting technology (FIG. 10).

The conjugation of the 6300 Dalton PNA to the 200,000 Dalton MAb-SA targeting system results in an increase in the effective molecular size of the imaging agent. This increase in size decreases systemic clearance 5-fold (Table 1) and blocks glomerular filtration and renal clearance of the PNA (Table 2). Conjugation of the PNA to the targeting vector re-directs the antisense agent from kidney to TfR-rich organs such as liver and spleen (Table 2) or brain (FIG. 8). The modest increase in PNA uptake in lung following conjugation to the TfR MAb is consistent with previous studies showing a modest uptake of TfR specific antibodies in this organ (Lee et al. (2000) J. Pharmacol. Exp. Ther., 292:1048-1052). There is little increase in uptake of the PNA by myocardium following conjugation to the targeting MAb (Table 2), as this organ is not targeted by TfR MAb's (Id.). The conjugation of the PNA to the targeting MAb results in an increase in both the BBB PS product and the plasma AUC, and both contribute to the >10-fold increase in brain uptake of the PNA following conjugation to the 8D3 MAb (FIG. 8). In contrast, the brain uptake of the unconjugated PNA is at the background level consistent with the absence of significant BBB transport of unconjugated PNAs across the BBB in vivo (Pardridge et al. (1995) Proc. Natl. Acad. Sci., USA, 92:5592-5596). Tyler et al (Tyler et al. (1999) Proc. Natl. Acad. Sci., USA, 96: 7053-7058) report that unconjugated PNAs do cross the BBB. However, a quantitative analysis of this study shows that the brain uptake of the unconjugated PNA is <0.0001% ID/g (Id.), which is >1000-fold lower than the brain uptake of the PNA/8D3 conjugate (FIG. 8). Given such a low level of brain uptake of the unconjugated PNA, it would not be possible to measure hybridization of a PNA radiopharmaceutical to a brain specific target mRNA in vivo.

In order to image target mRNA molecules in the brain with antisense radiopharmaceuticals, the antisense agent must be able to access transport pathways within the organ that mediate both the import and export of the antisense agent between the blood and organ compartments. No target mRNA can be imaged if there is no initial import from blood of the antisense radiopharmaceutical into the target organ. However, there must also be subsequent export of the antisense radiopharmaceutical back to blood from areas outside the region of interest in order to obtain a significant ratio in the signal and noise of the imaging modality. The BBB transferrin receptor (TfR) is a bi-directional transport system (Zhang and Pardridge 92001) J. Neurochem., 76: 1597-1600), and enables the receptor-mediated transcytosis of either transferrin (Tf) or a TfR MAb from blood to brain (Skarlatos et al. (1995) Brain Res., 683: 164-171). In addition, the BBB TfR also mediates the reverse transcytosis of either Tf or TfR MAbs from brain back to blood (Zhang and Pardridge 92001) J. Neurochem., 76: 1597-1600), as shown in FIG. 6A. The endogenous transport pathways for Tf at both the BBB and the brain cell membrane (BCM) allow for the sequential import of holo-transferrin from blood to brain followed by the export of apo-transferrin from brain back to blood (Id.). The TfR MAb traces these endogenous Tf transport pathways without interference in the transport of the endogenous Tf (Skarlatos et al. (1995) Brain Res., 683: 164-171). By 6 hours after intravenous injection of the PNA/8D3 conjugate, there is efflux back to the blood of >67% of the initial radioactivity imported into brain (FIG. 9). Therefore, subsequent brain scanning in the transgenic mice was performed at 6 hours after an intravenous injection of the PNA/8D3 conjugate. Another requisite for in vivo imaging of target mRNA with antisense radiopharmaceuticals is that the hybridization of the PNA to the target mRNA is not sterically inhibited by conjugation of the PNA to the targeting 8D3-SA vector. The in vitro studies demonstrated that conjugation of the PNA to the 8D3 MAb did not impair the ability of the PNA to hybridize to the target HD mRNA, based on either cell free translation or RNase protection assays (FIG. 7).

A conjugated PNA radiopharmaceutical that was enabled to access both import and export transport pathways between blood and brain (FIG. 6A), and which contained a specific base sequence, might be selectively sequestered in the brain of animals specifically expressing the target mRNA. This hypothesis is confirmed with the in vivo imaging studies performed in the control littermate mice and the HD exon 1 transgenic mice (FIG. 10). By 6 hours after intravenous injection, >⅔ of the initial radioactivity in brain has effluxed back to blood (FIG. 9), which is consistent with the reduced level of brain radioactivity in the littermate control mice at 6 hours (FIG. 10C). In contrast, the brain radioactivity at 6 hours is approximately 3-fold greater in the HD transgenic mice as compared to the littermate control mice (FIG. 10C). The brain scans of the transgenic mice show widespread sequestration of the imaging agent by the HD mRNA (FIG. 10A), consistent with the generalized expression of the HD exon 1 transcript in the brains of these transgenic mice (Kirupa et al. (1999) Phil. Trans. R. Soc. Lond., 354: 963-969).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of imaging in vivo expression of a gene in a brain cell of a vertebrate, said method comprising:
i) administering to said vertebrate an imaging reagent comprising a detectable label attached to a first nucleic acid that specifically hybridizes to a second nucleic acid transcribed from said gene, where said first nucleic acid is linked to a targeting ligand that binds a receptor on a cell comprising the blood brain barrier of said vertebrate and crosses said blood brain barrier; and
ii) detecting the presence or quantity of a signal produced by said detectable label in said brain cell where the presence or quantity of said label indicates the presence or quantity of a nucleic acid transcribed from said gene or cDNA.

2. The method of claim 1, wherein said nucleic acid is a peptide nucleic acid (PNA).

3. The method of claim 1, wherein said targeting ligand is selected from the group consisting of an antibody that specifically binds to a receptor on a cell comprising the blood brain barrier, and a substrate specifically bound by a receptor on a cell comprising the blood brain barrier.

4. The method of claim 3, wherein said targeting ligand is selected from the group consisting of insulin, transferrin, insulin-like growth factor I (IGF-I), insulin-like growth factor II (IGF-II), basic albumin, leptin, and prolactin.

5. The method of claim 3, wherein said targeting ligand is an antibody that specifically binds to a receptor selected from the group consisting of an insulin receptor, a transferrin receptor, an insulin-like growth factor I (IGF-IR) receptor, and insulin-like growth factor II receptor (IGF-IIR), and a leptin receptor.

6. The method of claim 1, wherein said first nucleic acid is linked to said targeting ligand by a linker or by an affinity tag.

7. The method of claim 1, wherein said first nucleic acid is linked to said targeting ligand by an affinity tag comprising a biotin and a molecule that binds to biotin.

8. The method of claim 7, wherein said molecule that binds to biotin is selected from the group consisting of an avidin, a streptavidin, and an anti-biotin antibody.

9. The method of claim 7, wherein said first nucleic acid is a peptide nucleic acid.

10. The method of claim 9, wherein the carboxyl terminal of said first nucleic acid is amidated.

11. The method of claim 7, wherein said first nucleic acid is an antisense peptide nucleic acid.

12. The method of claim 7, wherein said first nucleic acid bears a protecting group.

13. The method of claim 7, wherein said first nucleic acid is a peptide nucleic acid having an amidated carboxyl terminal.

14. The method of claim 1, wherein said detectable label is selected from the group consisting of an radioactive label, a magnetic label, a spin label, an enzymatic label, a colorimetric label, and a fluorescent label.

15. The method of claim 1, wherein said nucleic acid is labeled with a radiolabeled amino acid.

16. The method of claim 15, wherein said radiolabeled amino acid is a tyrosine labeled with 125⁻I.

17. The method of claim 15, wherein said radiolabeled amino acid is a lysine labeled with 111-indium.

18. The method of claim 1, wherein said gene is a gene that encodes a molecule selected from the group consisting of a receptor, and enzyme, a structural protein, and a transcription factor.

19. The method of claim 1, wherein:
said first nucleic acid is a peptide nucleic acid;
said targeting ligand is an antibody that specifically binds to a receptor on a cell comprising the blood-brain barrier; and
said first nucleic acid is attached to said targeting ligand through an affinity tag.

20. The method of claim 19, wherein said antibody is a monoclonal antibody.

21. The method of claim 20, wherein said imaging reagent comprises a radioactive label or a magnetic label.

22. The method of claim 21, wherein said first nucleic acid is labeled with a radiolabeled amino acid.

23. The method of claim 21, wherein said affinity tag is an affinity tag comprising a biotin.

24. The method of claim 23, wherein said antibody is a monoclonal antibody.

25. The method of claim 23, wherein said receptor is selected from the group consisting of a transferin receptor and an insulin receptor.

26. The method of claim 25, wherein said receptor is a transferrin receptor.

27. The method of claim 26, wherein the carboxyl terminal of said first nucleic acid is amidated.

28. The method of claim 1, wherein said contacting comprising systemically administering said imaging reagent to a living organism.

29. The method of claim 28, wherein said organism is a mammal.

30. The method of claim 28, wherein said organism is a non-human mammal.

31. The method of claim 28, wherein said organism is a human.

32. An imaging reagent for in vivo labeling of a gene or gene product, said imaging reagent comprising a detectable label attached to a first nucleic acid that specifically hybridizes to a second nucleic acid transcribed from said gene, where said first nucleic acid is linked to a targeting ligand that binds a receptor on a cell comprising the blood brain barrier of a vertebrate and crossing said blood brain barrier.

33. The reagent of claim 32, wherein said targeting ligand is selected from the group consisting of an antibody that specifically binds to a receptor on a cell comprising the blood brain barrier, and a substrate specifically bound by a receptor on cell comprising the blood brain barrier.

34. The reagent of claim 33, wherein said targeting ligand is selected from the group consisting of insulin, transferrin, insulin-like growth factor I (IGF-I), insulin-like growth factor II (IGF-II), basic albumin, leptin, and prolactin.

35. The reagent of claim 33, wherein said targeting ligand is an antibody that specifically binds to a receptor selected from the group consisting of an insulin receptor, a transferrin receptor, an insulin-like growth factor I (IGF-IR) receptor, and insulin-like growth factor II receptor (IGF-IIR), and a leptin receptor.

36. The reagent of claim 32, wherein said first nucleic acid is linked to said targeting ligand by a linker or by an affinity tag.

37. The reagent of claim 32, wherein said first nucleic acid is linked to said targeting ligand by an affinity tag comprising a biotin and a molecule that binds to biotin.

38. The reagent of claim 37, wherein said molecule that binds to biotin is selected from the group consisting of an avidin, a streptavidin, and an anti-biotin antibody.

39. The reagent of claim 37, wherein said first nucleic acid is a peptide nucleic acid.

40. The reagent of claim 39, wherein the carboxyl terminal of said first nucleic acid is amidated.

41. The reagent of claim 37, wherein said first nucleic acid is an antisense peptide nucleic acid.

42. The reagent of claim 37, wherein said first nucleic acid bears a protecting group.

43. The reagent of claim 37, wherein said first nucleic acid is a peptide nucleic acid having an amidated carboxyl terminal.

44. The reagent of claim 32, wherein said detectable label is selected from the group consisting of an radioactive label, a magnetic label, a spin label, an enzymatic label, a calorimetric label, and a fluorescent label.

45. The reagent of claim 32, wherein said nucleic acid is labeled with a radiolabeled amino acid.

46. The reagent of claim 45, wherein said radiolabeled amino acid is a tyrosine labeled with 125⁻I.

47. The reagent of claim 45, wherein said radiolabeled amino acid is a lysine labeled with 111-indium.

48. The reagent of claim 32, wherein said gene is a gene that encodes a molecule selected from the group consisting of a receptor, and enzyme, a structural protein, and a transcription factor.

49. The reagent of claim 32, wherein:
said first nucleic acid is a peptide nucleic acid;
said targeting ligand is an antibody that specifically binds to a receptor on a cell comprising the blood-brain barrier; and
said first nucleic acid is attached to said targeting ligand through an affinity tag.

50. The reagent of claim 49, wherein said antibody is a monoclonal antibody.

51. The reagent of claim 50, wherein said imaging reagent comprises a radioactive label or a magnetic label.

52. The reagent of claim 51, wherein said first nucleic acid is labeled with a radiolabeled amino acid.

53. The reagent of claim 51, wherein said affinity tag is an affinity tag comprising a biotin.

54. The reagent of claim 53, wherein said antibody is a monoclonal antibody.

55. The reagent of claim 53, wherein said receptor is selected from the group consisting of a transferrin receptor and an insulin receptor.

56. The reagent of claim 55, wherein said receptor is a transferrin receptor.

57. The reagent of claim 56, wherein the carboxyl terminal of said first nucleic acid is amidated.

58. A kit for in vivo imaging of a gene or a gene product in a brain cell, said kit comprising a container containing an imaging reagent comprising a detectable label attached to a first nucleic acid that specifically hybridizes to a second nucleic acid transcribed from said gene, where said first nucleic acid is linked to a targeting ligand that binds a receptor on a cell comprising the blood brain barrier of a vertebrate and crossing said blood brain barrier.

59. A kit for imaging a gene or a gene product in a brain cell, said kit comprising a container a nucleic acid that specifically hybridizes to said gene or to a nucleic acid transcribed from said gene attached to an affinity tag; and a container containing a targeting ligand that is capable of binding a receptor on a cell comprising the blood brain barrier of a vertebrate and crossing said blood brain barrier, where said targeting ligand is attached to an affinity tag such that when said nucleic acid is contacted to said targeting ligand the nucleic acid attaches to the targeting ligand by binding of the affinity tags.

60. The kit of claim 59, wherein said nucleic acid is a peptide nucleic acid.

61. The kit of claim 59, wherein said nucleic acid is labeled with a detectable label.


SEQUENCE LISTING (5 sequences)

SEQ ID NO: 1 - protein, 8 aa, Artificial Sequence
  epitope for epitope tag
DYKDDDDK

SEQ ID NO: 2 - DNA, 16 bp, Artificial Sequence
  HD-PNA
cttccattttaccaac

SEQ ID NO: 3 - DNA, 16 bp, Artificial Sequence
  complement of HD target mRNA
gttggtaaaatggaag

SEQ ID NO: 4 - DNA, 18 bp, Artificial Sequence
  peptide nucleic acid (PNA)
ctccgcttcttcctgcca

SEQ ID NO: 5 - DNA, 16 bp, Artificial Sequence
  anti-luciferase PNA
cttccattttaccaac